SEQUENCE LISTING (10 sequences)

SEQ ID NO: 1 - DNA, 27999 bp, synthetic construct
  misc_feature 1..27999: note = Recombinant polynucleotide
  source 1..27999: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatctacatgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactttttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaccggataatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataagcacaagctgatgagtccggatttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaccggattaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaaccggataaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 2 - DNA, 28719 bp, synthetic construct
  misc_feature 1..28719: note = Recombinant polynucleotide
  source 1..28719: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatctacatgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactttttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaccggataatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataagcacaagctgatgagtccggatttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaccggattaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatctagaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaaccggatatggtgagcaaaggagaagaggacaacatggcatcactcccagctacacatgagctgcatatcttcggatccatcaacggagtggacttcgatatggtgggacagggtacagggaacccaaacgacggatacgaggagttgaacctgaagagtaccaagggagacctgcagttctcaccatggatactcgtcccacatataggatacggctttcatcagtacctgccctatccagacggaatgtcacctttccaggcagccatggttgacgggagcggttaccaggtccacaggacaatgcagtttgaggacggagcctcattgaccgtgaactacagatatacctacgaaggaagccatatcaagggagaggctcaagtgaagggaactggattcccagcggacggacccgtgatgaccaacagtctgacggctgcagactggtgcagatccaaaaagacctacccaaatgacaagacaatcataagcaccttcaagtggtcatacactacaggaaacgggaagagatacaggagcactgccagaaccacatacactttcgccaagcctatggctgcaaactacctcaagaaccaacccatgtatgtgttcagaaagacagaactgaagcattctaagaccgaactgaacttcaaggagtggcagaaggcctttactgacgtgatgggaatggacgaactctacaagtaatgaccggataaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 3 - DNA, 28831 bp, synthetic construct
  misc_feature 1..28831: note = Recombinant polynucleotide
  source 1..28831: mol_type = other DNA
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatctacatgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactttttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaccggataatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaccggatttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtccggatttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaccggattaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaaccggataaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaa

SEQ ID NO: 4 - DNA, 719 bp, synthetic construct
  misc_feature 1..719: note = Recombinant nucleic acid
  source 1..719: mol_type = other DNA
  CDS 1..708
atggtgagcaaaggagaagaggacaacatggcatcactcccagctacacatgagctgcatatcttcggatccatcaacggagtggacttcgatatggtgggacagggtacagggaacccaaacgacggatacgaggagttgaacctgaagagtaccaagggagacctgcagttctcaccatggatactcgtcccacatataggatacggctttcatcagtacctgccctatccagacggaatgtcacctttccaggcagccatggttgacgggagcggttaccaggtccacaggacaatgcagtttgaggacggagcctcattgaccgtgaactacagatatacctacgaaggaagccatatcaagggagaggctcaagtgaagggaactggattcccagcggacggacccgtgatgaccaacagtctgacggctgcagactggtgcagatccaaaaagacctacccaaatgacaagacaatcataagcaccttcaagtggtcatacactacaggaaacgggaagagatacaggagcactgccagaaccacatacactttcgccaagcctatggctgcaaactacctcaagaaccaacccatgtatgtgttcagaaagacagaactgaagcattctaagaccgaactgaacttcaaggagtggcagaaggcctttactgacgtgatgggaatggacgaactctacaagtaattaattaa

SEQ ID NO: 5 - protein, 236 aa, synthetic construct
  REGION 1..236: note = Synthetic Construct
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYK

SEQ ID NO: 6 - DNA, 1650 bp, synthetic construct
  misc_feature 1..1650: note = Recombinant nucleic acid
  source 1..1650: mol_type = other DNA
  CDS 1..1650
atggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtataccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggtggtaagatcgccgtg

SEQ ID NO: 7 - protein, 550 aa, synthetic construct
  REGION 1..550: note = Synthetic Construct
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 8 - DNA, 513 bp, synthetic construct
  misc_feature 1..513: note = Recombinant nucleic acid
  source 1..513: mol_type = other DNA
  CDS 1..513
atggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcg

SEQ ID NO: 9 - protein, 171 aa, synthetic construct
  REGION 1..171: note = Synthetic Construct
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 10 - DNA, 29882 bp, Coronavirus
  source 1..29882: mol_type = other DNA
attaaaggtttataccttcccaggtaacataccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactttttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaa

(19) United States
(12) Patent Application Publication
(10) Pub. No.: US 2025/0161431 A1
(43) Pub. Date: May 22, 2025

(54) ATTENUATED SARS-COV-2

(71) Applicant: Board of Regents, The University of Texas System, Austin, TX (US)

(72) Inventors: Xuping XIE, Galveston, TX (US); Pei-yong SHI, Galveston, TX (US)

(21) Appl. No.: 18/833,989

(22) PCT Filed: Dec. 9, 2022
(86) PCT No.: PCT/US2022/052405

§ 371 Date: Jul. 29, 2024

Related U.S. Application Data
(60) Provisional application No. 63/309,361, filed on Feb. 11, 2022.

(51) Int. Cl. A61K 39/215; A61K 39/00; A61P 31/14; C12N 7/00; C12Q 1/02

(52) U.S. Cl. CPC A61K 39/215; A61P 31/14; C12N 7/00; C12Q 1/025; A61K 2039/5254; C12N 2770/20021; C12N 2770/20022; C12N 2770/20034; G01N 2333/165; G01N 2500/10

(57) ABSTRACT

This composition of this invention is comprised of live attenuated SARS-CoV-2 constructs as vaccines or research tools. Described herein is a highly attenuated SARS-CoV-2 with deleted accessory proteins and modified transcriptional regulator sequences (TRS) that can serve as a live-attenuated vaccine platform and a BSL-2 experimental system. Certain embodiments are directed to a live attenuated SARS-CoV-2 having a modified transcriptional regulatory sequence (TRS) and a deletion of one or more open reading frames selected from ORF3a, ORF3, ORF6, ORF7, and/or ORFS.

### RELATED APPLICATIONS

This application is an International Application claiming priority to U.S. Provisional Application 63/309,361 filed Feb. 11, 2022, which is incorporated herein by reference in its entirety.

### STATEMENT REGARDING FEDERALLY FUNDED RESEARCH

None.

### REFERENCE TO SEQUENCE LISTING

A sequence listing in ST.26 XML file is being submitted concurrently and electronically with this application. The sequence listing is incorporated herein by reference.

### BACKGROUND

The pandemic of SARS-CoV-2 has caused over 342 million confirmed infections and 5.6 million deaths (as of Jan. 21, 2022; URL coronavirus.jhu.edu/). Different vaccine platforms have been successfully developed for COVID-19 at an unprecedented pace, including mRNA, viral vector, subunit protein, and inactivated virus. Live-attenuated vaccines of SARS-CoV-2 have not been actively explored, even though they may have advantages of low cost, strong immunity, and long immune durability. The SARS-CoV-2 virion consists of an internal nucleocapsid, formed by the genomic RNA coated with nucleocapsid (N) proteins, and an external envelope, formed by a cell-derived bilipid membrane embedded with spike (S), membrane (M), and envelope (E) proteins (Yao et al., Cell 183, 730-738 e713, 2020). The plus-sense, single-stranded viral RNA genome encodes open-reading-frames (ORFs) for replicase (ORF1a/ORF1b), S, E, M, and N structural proteins, as well as seven additional ORFs for accessory proteins (Hu et al., Nat Rev Microbiol, 2020). Although the exact functions of SARS-CoV-2 accessory proteins remain to be determined, previous studies of other coronaviruses suggest that these proteins are not essential for viral replication but can modulate replication and pathogenesis through interacting with host pathways (Comar et al., mBio 10, 2019; Thornbrough et al., mBio 7, e00258, 2016; Nakagawa et al., J Virol 92, 2018; Niemeyer et al., J Virol 87, 12489-95, 2013; Rabouw et al., PLoS Pathog 12, e1005982, 2016). Thus, deletion of the accessory proteins could be used to attenuate SARS-CoV-2.

Reverse genetic systems are important tools to engineer and study viruses. In response to the COVID-19 pandemic, three types of reverse genetic systems have been developed for SARS-CoV-2: (i) an infectious cDNA clone (Xie et al., Cell Host Microbe 27, 841-48 e843, 2020; Xie et al., Nat Commun 11, 5214, 2020; Mulligan et al., Nature 586, 589-93, 2020; Hou et al., Cell 182, 1-18, 2020; Thao et al., Nature 582, 561-65, 2020), (ii) a transient replicon (a self-replicating viral RNA with one or more genes deleted)(Xia et al., Cell Rep 33, 108234, 2020; Kotaki et al., Sci Rep 11, 2229, 2021), and (iii) a trans-complementation system (replicon RNAs in cells that express the missing genes in the replicon) (Zhang et al., Cell 184, 2229-38 e2213, 2021; Ricardo-Lax et al., Science 374, 1099-106, 2021; Ju et al., PLoS Pathog 17, e1009439, 2021). The three systems have their own strengths and weaknesses and are complementary to each other when applied to address different research questions. The infectious cDNA clone requires biosafety level-3 (BSL-3) containment to recover and handle infectious SARS-CoV-2. The transient replicon system requires RNA preparation and transfection for each experiment; cell lines harboring replicons that can be continuously cultured, like those developed for hepatitis C virus and other plus-sense RNA viruses (Lohmann et al., Science 285, 110-13, 1999; Khromykh and Westaway, J. Virol. 71, 1497-505, 1997; Shi et al., Virology 296, 219-33, 2002), have yet to be established for SARS-CoV-2. The trans-complementation system produces virions that can infect naïve cells for only a single round. Compared with the infectious cDNA clone, both the replicon and trans-complementation system have the advantage of allowing experiments to be performed at BSL-2. A new system that combines the strengths of the current three systems (e.g., multiple rounds of viral infection of naïve cells that can be performed at BSL2) will be useful for COVID-19 research and countermeasure development.

### SUMMARY

The need for additional live attenuated SARS-CoV-2 constructs as vaccines or research tools is addressed by the compositions described herein. Described herein is a highly attenuated SARS-CoV-2 (with deleted accessory proteins and modified transcriptional regulator sequences (TRS)) that can serve as a live-attenuated vaccine platform and a BSL-2 experimental system. Certain embodiments are directed to a live attenuated SARS-CoV-2 having a modified transcriptional regulatory sequence (TRS) and a deletion of one or more open reading frames selected from ORF3a, ORF3, ORF6, ORF7, and/or ORF8. In certain aspects the attenuated recombinant SARS-CoV-2 has a genome having (i) a transcriptional regulatory sequences (TRS) comprising a nucleotide sequence of CCGGAT and (ii) a deletion of open reading frames 3, 6, 7, and 8. In certain aspects the nucleic acid segment encoding the attenuated recombinant SARS-CoV-2 has a nucleic acid sequence that is at least 98% to 100% identical to the nucleic acid sequence of SEQ ID NO:1, SEQ ID NO:2, or SEQ ID NO:3. In certain aspects the SARS-CoV-2 is a variant SARS-CoV-2. The attenuated recombinant SARS-CoV-2 can further comprise a heterologous S protein, e.g., a SARS-CoV-2 variant S protein. The heterologous S protein can include any combination of substitution(s), insertion(s) or deletion (2) including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more amino acid substitutions or variants; a 1, 2, 3, 4, 5 deletion of consecutive amino acids, and/or a 1, 2, 3, 4, 5, amino acid insertion with respect to the S protein of SEQ ID NO:1, SEQ ID NO:2 or SEQ ID NO:3. Thus, the heterologous S protein can be an S protein variant. In certain aspects the S protein variant can be a S protein variant selected from USA-WA1/2020 spike, D614G-spike, XD-spike, Alpha-spike, Belt-spike, Delta-spike, BA.1-spike, BA.2-spike, BA.3-spike, BA.4/5-spike, BA.4/6-spike, BA.2.12.1-spike, BA.2.75-spike, BA.2.75.2-spike, BF7-spike, XBB.1-spike, BQ.1-spike, BQ.1.1-spike, BJ.1-spike, or BA.2.10.4-spike.

The attenuated recombinant SARS-CoV-2 can be in an expression cassette. The expression cassette can be included in a plasmid backbone. The attenuated recombinant SARS-CoV-2 can include a nucleic acid segment encoding a reporter protein. In certain aspects the nucleic acid segment encoding the reporter protein is inserted into the SARS-CoV-2 genome or nucleic acid between the segments encoding the N and M proteins. The reporter protein can be a fluorescent or luminescent protein. In certain aspects the fluorescent protein is mNeonGreen protein. In certain aspects the luminescent protein is nanoluciferase protein. The reporter protein can be, for example but not limited to mNeonGreen (e.g., SEQ ID NO:4 and SEQ ID NO:5) or nanoluciferase reporter (e.g., SEQ ID NO:6 and SEQ ID NO:7)). The recombinant SARS-CoV-2 can be encoded by SEQ ID NO:1, SEQ ID NO:2, or SEQ ID NO:3. The recombinant SARS-CoV-2 nucleic acid segment can be at least 95, 96, 97, 98, 99, to 100% identical to the nucleic acid sequence of SEQ ID NO:1, SEQ ID NO:2, or SEQ ID NO:3. In certain aspects an expression cassette is comprised in a plasmid backbone. The SARS-CoV-2 nucleic acid segment can be operatively coupled to a heterologous promoter segment.

Other embodiments are directed to a host cell comprising the attenuated recombinant SARS-CoV-2 described herein.

Still other embodiments are directed to vaccine compositions comprising the attenuated recombinant SARS-CoV-2 described herein.

Embodiments are directed to assays for SARS-CoV-2 replication comprising: contacting a cultured cell expressing or containing a SARS-CoV-2 nucleotide sequence of any one of claims 1 to 10 forming a test cell; contacting the test cell with a test agent; and assessing the replication of the SARS-CoV-2 in the presence of the test agent. The cultured cell can be a Vero cell. The cultured cell(s) can be assayed in a multi-well plate. The multi-well plate can be a 96 well microtiter plate. In certain aspects the cultured cells are incubated for about 12, 24, 36, or 48 hours before measuring the reporter signal.

Certain embodiments are directed to host cells comprising an expression cassette as described herein or a recombinant SARS-CoV-2 RNA transcribed from an expression cassette described herein.

Other embodiments are directed to recombinant SARS-CoV-2 genomes comprising a a reporter protein. The reporter protein can be a fluorescent or luminescent protein, or other detectable polypeptide or transcript. In certain aspects the fluorescent protein is a mNeonGreen protein. In certain aspects the luminescent protein is a nanoluciferase protein.

Other embodiments are directed to assays for SARS-CoV-2 replication comprising: (i) contacting a cultured cell expressing or containing a SARS-CoV-2 nucleotide sequence as described herein forming a test cell; (ii) contacting the test cell with a test agent; and (iii) assessing the replication of the SARS-CoV-2 in the presence of the test agent. In certain aspects the cultured cell is a Vero cell. The cultured cell can be assayed in a multi-well plate. In certain aspects the multi-well plate is a 96 well microtiter plate. The cells can be incubated for about 12, 24, 36, or 48 hours before measuring the reporter signal.

The term “coronavirus” refers to a virus whose genome is plus-stranded RNA of about 27 kb to about 33 kb in length depending on the particular virus. The virion RNA has a cap at the 5′ end and a poly A tail at the 3′ end. The length of the RNA makes coronaviruses the largest of the RNA virus genomes. Coronavirus RNAs can encode: (1) an RNA-dependent RNA polymerase; (2) N-protein; (3) three envelope glycoproteins; and (4) three non-structural proteins. These coronaviruses infect a variety of mammals and birds. They cause respiratory infections (common), enteric infections (mostly in infants >12 mo.), and possibly neurological syndromes. Coronaviruses are transmitted by aerosols of respiratory secretions. Coronaviruses are exemplified by, but not limited to, human enteric SARS-CoV-2 (WA1/2020 strain (SEQ ID NO:10, provided as a reference sequence)), (GenBank accession number NC_045512.2), coV (ATCC accession #VR-1475), human coV 229E (ATCC accession #VR-740), human coV OC43 (ATCC accession #VR-920), and SARS-coronavirus (Center for Disease Control).

The term “nucleic acid” refers to a polymeric compound comprising nucleosides or nucleoside analogs which have nitrogenous heterocyclic bases or base analogs linked together to form a polynucleotide, including conventional RNA, DNA, mixed RNA-DNA, and polymers that are analogs thereof. A nucleic acid “backbone” may be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid may be ribose, deoxyribose. Nitrogenous bases may be conventional bases (A, G, C, T, U), analogs thereof (e.g., inosine or others; see The Biochemistry of the Nucleic Acids 5-36, Adams et al., ed., 11th ed., 1992).

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins.

The term “recombinant” refers to an artificial combination of two otherwise separated segments of nucleic acid, e.g., by chemical synthesis or by the manipulation of isolated segments of nucleic acids by genetic engineering techniques.

The term “SARS-CoV-2 replicon” is used to refer to a nucleic acid molecule expressing SARS-CoV-2 genes such that it can direct its own replication (amplification).

The term “SARS-CoV-2 replicon particle” refers to a virion or virion-like structural complex incorporating a SARS-CoV-2 replicon.

The term “SARS-CoV-2 reporter virus” refers to a virus that is capable of directing the expression of a sequence(s) or gene(s) of interest. The reporter construct can include a 5′ sequence capable of initiating transcription of a nucleic acid encoding a reporter molecule or protein such as luciferase, fluorescent protein, Neo, SV2 Neo, hygromycin, phleomycin, histidinol, and DHFR. The reporter virus can be used an indicator of infection of a cell by a SARS-CoV-2 virus.

The term “variant” refers to a viral genome or portion of viral genome or its encoded proteins having a change in its genetic material or expressed polypeptide, in particular a change (i.e., deletion, substitution, or insertion) relative to a reference nucleotide or amino acid sequence. Polypeptide sequence variants may share at least 50%, 55%, 60%, 65%, 70%, 75%, 80%, 85%, 90%, 95%, 96%, 97%, 98% or 99% sequence identity. Polypeptides may possess deletions, insertions, and/or substitutions of amino acids relative to the reference sequence. Polypeptides with (i) amino acid substitution(s); (ii) amino acid deletion(s); (iii) amino acid insertion(s) (added amino acids), (iv) amino acid substitution(s) and deletion(s); (v) amino acid substitution(s) and insertion(s); (vi) amino acid deletion(s) and insertion(s); or (vii) substitution(s), deletion(s) and substitutions.

In certain aspects, the term “SARS-CoV-2 variant” or SARS-CoV-2 S protein variant contains at least one or more of the following mutations in the spike protein: D614G; D936Y; P1263L; L5F; N439K; R21I; D839Y; L54F; A879S; L18F; F1121L; R847K; T478I; A829T; Q675H; S477N; H49Y; T29I; G769V; GI 124V; V1176F; K1073N; P479S; S1252P; Y145 deletion; E583D; R214L; A1020V; Q1208H; D215G; H146Y; S98F; T95I; G1219C; A846V; 1197V; R102I; V367F; T572I; A1078S; A831V; P1162L; T73I; A845S; G1219V; H245Y; L8V; Q675R; S254F; V483A; Q677H; D138H; D80Y; M1237T; Dl146H; E654D; H655Y; S50L; S939F; S943P; G485R; Q613H; T76I; V341I; M153I; S221L; T859I; W258L; L242F; P681L; V289I; A520S; V1104L; V1228L; L176F; M1237I; T307I; T716I; L141; M1229I; A1087S; P26S; P330S; P384L; R765L; S940F; T323I; V826L; E1202Q; L1203F; L611F; V615I; A262S; A522V; A688V; A706V; A892S; E554D; Q836H: T1027I; T22I; A222V; A27S; A626V; C1247F; K1191N; M731I; P26L; S1147L; S1252F; S255F; V1264L; V308L; D80A; 1670L; P251L; P631S;*I274Q; A344S; A771S; A879T; D1084Y; D253G; H1101Y; L1200F; Q14H; Q239K; A623V; D215Y; E1150D; G476S; K77M; M177I; P812S; S704L; T51I; T547I; T791I; V1122L; Y145H; D574Y; G142D; G181V; 1834T; N370S; P812L; S12F; T791P; V90F; W152L; A292S; A570V; A647S; A845V; D1163Y; G181R; L84I; L938F; P1143L; P809S; R78M; T1160I; V1133F; V213L; V615F; A831V; D839Y; D839N; D839E; S943P; P1263L; or V622F; and combinations thereof.

The term “expression vector” refers to a nucleic acid that is capable of directing the expression of a sequence(s) or gene(s) of interest. The vector construct can include a 5′ sequence capable of initiating transcription of a nucleic acid, e.g., all or part of a SARS-CoV-2 virus. The vector may also include nucleic acid molecule(s) to allow for production of virus, a 5′ promoter that is capable of initiating the synthesis of viral RNA in vitro from cDNA, as well as one or more restriction sites, and a polyadenylation sequence. In addition, the constructs may contain selectable markers such as Neo, SV2 Neo, hygromycin, phleomycin, histidinol, and DHFR. Furthermore, the constructs can include plasmid sequences for replication in host cells and other functionalities known in the art. In certain aspects the vector construct is a DNA construct.

The term “expression cassette” refers to a nucleic acid segment capable of directing the expression of one or more nucleic acids, or one or more nucleic acids that are in turn translated into an expressed protein.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1A-1G. Attenuation of Δ3678 SARS-CoV-2 in cell culture. (A) Scheme diagram for the construction of Δ678 (SEQ ID NO:3) and Δ3678 SARS-CoV-2 (SEQ ID NO:1). The deletions were introduced to the backbone of USA-WA1/2020 strain (SEQ ID NO:10). T7, T7 promoter; L, leader sequence; TRS, transcription regulatory sequences; ORF, open reading frame; E, envelope glycoprotein gene; M, membrane glycoprotein gene; N, nucleocapsid gene; UTR, untranslated region; pA, poly A tails. (B) Plaque morphologies of recombinant WT, Δ678, and Δ3678 viruses. Plaque assays were performed on Vero-E6 cells and stained on day 2.5 post-infection. (C-E) Replication kinetics of WT, Δ678, and Δ3678 SARS-CoV-2s on Vero-E6 (C), Calu-3 (D), and HAE (E) cells. WT, Δ678, and Δ3678 viruses were inoculated onto Vero-E6, Calu-3, and HAE cells at MOIs of 0.01, 0.1, and 2, respectively. After a 2-h incubation, the cells were washed three times with DPBS and continuously cultured under fresh 2% FBS DMEM. Culture supernatants were measured for infectious virus titers using plaque assays on Vero-E6 cells. (F) Intracellular levels of WT, Δ678, and Δ3678 RNA in HAE cells on day 7 post-infection. The HAE cells were washed with PDBS for three times, lysed by Trizol for RNA isolation, quantified for viral RNAs using RT-qPCR. Dots represent individual biological replicates (n=3 for Vero-E6 and Calu-3; n=5 for HAE). The values in the graph represent the mean±standard deviation. An unpaired two-tailed t test was used to determine significant differences between WT and Δ678/Δ3678 groups. P values were adjusted using the Bonferroni correction to account for multiple comparisons. Differences were considered significant if p<0.025; P<0.025, *: P<0.005, **; and P<0.0005, ***. (G) mNG-positive HAE cells after infection with mNG WT or mNG Δ3678 virus at an MOI of 0.5. Scale bar, 100 μm.

FIG. 2A-2G. Attenuation of Δ3678 SARS-CoV-2 in hamsters. (A) Experimental scheme of Δ3678 virus immunization and WT virus challenge. Hamsters were intranasally (I.N.) inoculated with 10⁶ PFU of WT or Δ3678 virus. On day 2 post-inoculation, organ viral loads (n=5) were measured by plaque assays on Vero-E6 cells. Nasal washes and oral swabs (n=10) were collected on days 2, 4, and 7 post-inoculation. On day 28 post-immunization, the hamsters were challenged by 10⁵ PFU of WT SARS-CoV-2. On days 2 and 4 post-challenge, plaque assays were performed to measure organ viral loads (n=5). On day 21 post-challenge, the animals were terminated to measure neutralization titer (NT₅₀). (B) Weight changes of hamsters after intranasal infection with WT (n=9) or Δ3678 (n=9) SARS-CoV-2. Uninfected mock group (n=9) was included as a negative control. Body weights were measured daily for 14 days. The data are shown as mean±standard deviation. The weight changes between Δ3678 and mock or WT groups were analyzed using two-factor analysis of variance (ANOVA) with Tukey's post hoc test. The black and red asterisks stand for the statistic difference between Δ3678 and mock or WT, respectively. *, P<0.05; **, P<0.01; ***, P<0.001. (C) Disease of Δ678 and Δ3678 virus-infected animals. The diseases include ruffled fur, lethargic, hunched posture, and orbital tightening. The percentages of animals with or without diseases are presented. (D-F) Viral loads in nasal wash (D), oral swab (E), trachea, and lung (F) after infection with Δ3678 or WT virus. Dots represent individual animals (n=5). The mean f standard error is presented. A non-parametric two-tailed Mann-Whitney test was used to determine the differences between mock, Δ3678, or WT groups. P values were adjusted using the Bonferroni correction to account for multiple comparisons. Differences were considered significant if p<0.025. *, P<0.025; **, P<0.005; ***, P<0.0005. (G) Neutralization titers of sera from WT- and Δ3678 virus-inoculated hamsters on days 7, 14, 21, and 28 post-inoculation. The neutralization titers were measured against WT SARS-CoV-2.

FIG. 3A-3J. Protection of A 3678 virus-immunized hamsters from WT SARS-CoV-2 challenge and transmission. (A, B) Weight loss (A) and diseases (B) of immunized and challenged hamsters. (A) Mock-immunized (n=5), Δ3678 virus-immunized (n=5), and WT virus-inoculated (n=5) hamsters were challenged with 105 PFU of WT SARS-CoV-2. The body weights were measured daily for 14 days post-challenge. The data are shown as mean±standard deviation. The weight changes between Δ3678- and mock- or WT-inoculated groups were statistically analyzed using two-factor analysis of variance (ANOVA) with Tukey's post hoc test. No statistical difference was observed between the Δ3678- and WT-inoculated groups. The statistic difference between the Δ3678- and mock-immunized groups are indicated. **, P<0.01; ***, P<0.001. (B) After the challenge, animals developed diseases, including ruffled fur, lethargic, hunched posture, and orbital tightening. The percentages of animals with or without diseases were presented. (C-F) Viral loads in the nasal wash (C), oral swab (D), trachea (E), and lung (F) after challenge. Dots represent individual animals (n=5). The values of mean f standard error of the mean are presented. A non-parametric two-tailed Mann-Whitney test was used to determine the statistical differences between Δ3678-immunized and mock-immunized or WT-inoculated groups. P values were adjusted using the Bonferroni correction to account for multiple comparisons. Differences were considered significant if p<0.025. *, P<0.025; **, P<0.005. (G) Neutralization titers of immunized hamsters before and after challenge. The “before challenge” sera were collected on day 28 post-immunization. The “after challenge” sera were collected on day 21 post-challenge. (H) Experimental design of transmission blockage in hamsters. Hamsters were immunized with 105 PFU of Δ3678 virus (n=5) or medium mock (n=5). In day 28 post-immunization, the hamsters were challenged with 10⁴ PFU of WT SARS-CoV-2; these animals served as transmission donors. On day 1 post-challenge, the donor hamsters were co-housed with clean recipient hamsters for 8 h. The nasal washes of donor hamsters were collected immediately after contact (i.e., 32 h post-challenge). The nasal washes of recipient hamsters were collected on days 2, 4, 6, and 8 post-contact. (I-J) Weight loss of donors post-challenge (I) and recipients post-contact (J). The data are shown as mean f standard deviation. The weight changes were statistically analyzed using two-factor analysis of variance (ANOVA) with Tukey's post hoc test. *, P<0.05; **, P<0.01; ***, P<0.001. (k) Viral loads in nasal wash of donors post-challenge and recipients post-contact. Dots represent individual animals (n=5). The values in the graph represent the mean±standard error of mean. A non-parametric two-tailed Mann-Whitney test was used to analyze the difference between the mock-immunized-and-challenged and Δ3678-immunized-and-challenged hamsters. **, P<0.01.

FIG. 4A-4G. Attenuation of Δ3678 SARS-CoV-2 in K18-hACE2 mice. (A) Experimental scheme. K-18-hACE2 mice were intranasally inoculated with 4, 40, 400, 4,000, or 40,000 PFU of WT (n=10) or Δ3678 virus (n=10). Lung viral loads were measured on days 2, 4, and 7 post-infection. The infected mice were monitored for body weight (B, C), disease (D, E), and survivals (F, G) for 14 days. The data are shown as mean±standard deviation. (B, C) Bodyweight changes. Different viral infection doses are indicated by different colors. The weight changes between mock- and virus-infected groups were statistically analyzed using two-factor analysis of variance (ANOVA) with Tukey's post hoc test. The green and brown asterisks indicate the statistical difference between mock and 4,000- or 40,000-PFU infection groups. **, P<0.01; ***, P<0.001. (D, E) Disease. The diseases include ruffled fur, lethargic, hunched posture, or orbital tightening. The percentages of hamsters with or without diseases were presented. (F, G) Survival. A mixed-model ANOVA using Dunnett's test for multiple comparisons was used to evaluate the statistical significance. h, Lung viral loads from WT- and Δ3678K-infected K18-hACE2 mice. Dots represent individual animals (n=10). The mean t standard error of mean is presented. A non-parametric two-tailed Mann-Whitney test was used to determine statistical significance. P values were adjusted using the Bonferroni correction to account for multiple comparisons. Differences were considered significant if p<0.025. ***, P<0.0005.

FIG. 5A-5F. ORF3a deletion is mainly responsible for the attenuation of Δ3678 virus through interfering with STAT1 phosphorylation during type-I interferon signaling. (A, B) Analysis of individual ORFs in BALB/c mice. (A) Experimental design. A mouse-adapted SARS-CoV-2 (MA) was used to construct individual ORF-deletion viruses. BALB/c mice were intranasally infected with 10⁴ PFU of WT and individual OFR deletion viruses (n=10 per virus) and quantified for lung viral loads on day 2 post-infection. (B) Lung viral loads from mice infected with different ORF deletion viruses. Dots represent individual animals (n=10). The mean f standard error of mean is presented. A non-parametric two-tailed Mann-Whitney test was used to determine the statistical difference between the WT and ORF deletion groups. P values were adjusted using the Bonferroni correction to account for multiple comparisons. Differences were considered significant if p<0.0083. *, P<0.0083; **, P<0.0017; ***, P<0.00017. (C-E) Replication kinetics of Δ3a virus in Vero-E6 (C), Calu-3 (D), and HAE (E) cells. The WT-MA and Δ3a were inoculated onto Vero-E6, Calu-3 and HAE cells at an MOI of 0.01, 0.1, and 2, respectively. After 2 h of incubation, the cells were washed three times with DPBS, continuously cultured with fresh 2% FBS DMEM, and quantified for infectious viruses in culture fluids at indicated time points. Dots represent individual biological replicates (n=3). The values represent the mean±standard deviation. An unpaired two-tailed t test was used to determine significant differences *, P<0.05; **, P<0.01; ***, P<0.001. (F) ORF3a deletion increases ISG expression in Δ3a-infected A549-hACE2 cells. A549-hACE2 cells were infected with WT or Δ3a virus at an MOI of 1 for 1 h, after which the cells were washed twice with PBS and continuously cultured in fresh medium. Intracellular RNAs were harvested at 24 h post-infection. Viral RNA copies and mRNA levels of IFN-α, IFITM1, ISG56, OAS1, PKR, and GAPDH were measured by RT-qPCR. The housekeeping gene GAPDH was used to normalize the ISG mRNA levels. The mRNA levels are presented as fold induction over mock samples. As a positive control, uninfected cells were treated with 1,000 units/ml IFN-α for 24 h. Dots represent individual biological replicates (n=6). The data represent the mean f standard deviation. An unpaired two-tailed t test was used to determine significant differences between Δ3a and WT-MA or IFN(+) groups. P values were adjusted using the Bonferroni correction to account for multiple comparisons. *, P<0.025: **, P<0.005: ***, P<0.0005. g, ORF3a suppresses type-I interferon by reducing STAT1 phosphorylation. A549-hACE2 cells were pre-treated with or without 1,000 units/ml IFN-α for 6 h. The cells were then infected with WT-MA or Δ3a virus at an MOI of 1 for 1 h. The infected cells were washed twice with PBS, continuously cultured in fresh media with or without 1,000 units/ml IFN-α, and analyzed by Western blot at 24 h post-infection.

FIG. 6A-6H. Development of mNG Δ 3678 virus for high-throughput neutralization and antiviral testing. (A) Genome structure of mNG Δ3678 SARS-CoV-2. The mNG gene was inserted between M and N genes. (B) mNG foci of Vero-E6-TMPRSS2 cells that were infected with mNG Δ3678 SARS-CoV-2 for 16 h. (C) Representative FFRNT neutralization curves for a COVID-19 antibody-positive and -negative serum. (D) Correlation between FFRNT₅₀ with PRNT₅₀ values of 20 COVID-19 convalescent sera. The Pearson correlation efficiency and P value are shown. (E) Scatter-plot of FFRNT₅₀/PRNT₅₀ ratios. The geometric mean is shown. Error bar indicates the 95% confidence interval of the geometric mean. (F) Inhibition of mNG-positive cells by remdesivir. A549-hACE2 cells were infected with mNG Δ3678 SARS-CoV-2 in the presence and absence of 10 μM remdesivir. The antiviral activity was measured at 24 h post-infection. (G) Antiviral response curve of remdesivir against mNG Δ3678 SARS-CoV-2. The calculated 50% effective concentration (EC₅₀) is indicated. Error bars indicate the standard deviations from four technical replicates. (H) The Dose response curve of a monoclonal antibody IgG14. A549-hACE2 cells were infected with Δ3a virus in the presence of different concentrations of IgG14. The mNG signals at 24 h post-infection were used to calculate the NT₅₀.

FIG. 7. Negative-staining electron microscopic images of WT, Δ678, and Δ3678 SARS-CoV-2s. Scale bar, 20 nm.

FIG. 8A-8B. Brightfield images of the cytopathic effects of WT, Δ678, and Δ3678 SARS-CoV-2-infected Vero-E6 and Calu-3 cells. The Vero-E6 and Calu-3 cells were infected with WT, Δ678, or Δ3678 virus at an MOI of 0.1 and 1.0, respectively. The images of infected Vero-E6 (A) and Calu-3 cells (B) were taken at 24 and 48 h post-infection, respectively. Scale bar, 100 μm.

FIG. 9A-9F. Lung pathology of Δ3678 virus-immunized and WT SARS-CoV-2-challenged hamsters. (A) Lung sections show typical interstitial pneumonia with moderate to severe inflammatory changes in mock-immunized and WT virus-challenged animals (mock-WT) or in WT virus-inoculated and WT virus-challenged animals (WT-WT). Reduced inflammatory changes are observed in Δ3678 virus-immunized and WT virus-challenged animals (Δ3678-WT). (B) Higher magnification images show large inflammatory cells in the airways and prominent septal thickening in the mock-WT and WT-WT groups. Such changes are minimal or absent in the Δ3678-WT group. (C-F) Comparative pathology scores calculated based on the criteria described in Table 1 below. The Δ3678-WT group shows a significant reduction in total pathology score (C), extent of inflammation (D), alveolar septa changes (E), and airway changes (F). Dots represent individual animals (n=5). The values of mean f standard error of mean are presented. A non-parametric two-tailed Mann-Whitney test was used to determine the statistical differences between Δ3678-WT and mock-WT or WT-WT groups. P values were adjusted using the Bonferroni correction to account for multiple comparisons. *, P<0.025.

FIGS. 10A-10C. Dose range immunization of Δ3678 virus to protect hamsters from WT SARS-CoV-2 challenge. (A) Weight loss of hamsters immunized with four different doses of Δ3678 virus. Hamsters were intranasally inoculated with 10², 10³, 10⁴, or 10⁵ PFU of Δ3678 virus (n=5 per dose). Body weights were measured for 14 days post-inoculation. The data are shown as mean±standard deviation. The weight changes were statistically analyzed using two-factor analysis of variance (ANOVA) with Tukey's post hoc test. No statistic difference was observed among mock and all Δ3678 dose groups. (B) Nasal viral loads in Δ3678 virus-immunized hamsters on days 2, 4, and 7 post-immunization. (C) Viral loads in nasal wash, oral swab, trachea, and lung from Δ3678-immunized and WT virus-challenged hamsters. The Δ3678-immunized hamsters were challenged with WT SARS-CoV-2 on day 28 post-immunization. The viral loads were measured on day 2 post-challenge. (B, C) Dots represent individual animals (n=5). The values in the graph represent the mean±standard error of mean. Dash lines indicate assay detection limitations. A non-parametric two-tailed Mann-Whitney test was used to determine the statistical differences between mock- and Δ3678-immunized hamsters. P values were adjusted using the Bonferroni correction to account for multiple comparisons.

FIGS. 11A-11C. Genetic stability analysis of Δ3678 SARS-CoV-2. (A) Experimental scheme. Passage 0 (P0) Δ3678 virus was divided into three T25 flasks for five rounds of independent passaging on Vero-E6 cells. (B) Plaque morphologies of P0 and P5 Δ3678 virus. (C) Mutations recovered from three independently cultured P5 Δ3678 viruses. The P5 Δ3678 viral RNAs were extracted and amplified by RT-PCR. Whole-genome sequencing was performed on the RT-PCR products. Mutations from the P5 viruses were annotated with amino acid changes and specific genes.

FIG. 12A-12B. Construction of mouse-adapted SARS-CoV-2s with individual ORF deletions. (A) Mouse-adapted SARS-CoV-2 genome. Mouse-adapted SARS-CoV-2 (MA-SARS-CoV-2) contains three mutations in spike glycoprotein: K417E, N501Y, and Q613R. These mutations confer SARS-CoV-2 to replicate in BALB/c mice. Open reading frames, ORFs; E, envelope glycoprotein gene; L, leader sequence; M, membrane glycoprotein gene; N, nucleocapsid gene; UTR, untranslated region. (B) Plaque morphologies of MA-SARS-CoV-2s with individual ORF deletions. All these ORF deletion viruses were constructed in the backbone of MA-SARS-CoV-2. Plaque assays were performed on Vero-E6 cells and stained on day 2.5 post-infection.

FIG. 13A-13D. Neutralization of Omicron sublineages before and after 4 doses of mRNA vaccine. (A) Construction of Omicron sublineage-spike mNG SARS-CoV-2. mNG USA-WA1/2020 SARS-CoV-2 was used to engineer Omicron-spike SARS-CoV-2s. The mNG reporter gene was engineered at the open-reading-frame-7 (ORF7) of the USA-WA1/2020 genome. 1 Amino acid mutations, deletions (Δ), and insertions (Ins) are indicated for variant spikes in reference to the USA-WA1/2020 spike. L: leader sequence; ORF: open reading frame; NTD: N-terminal domain of S1; RBD: receptor binding domain of S1; S: spike glycoprotein; S1: N-terminal furin cleavage fragment of S; S2: C-terminal furin cleavage fragment of S; E: envelope protein; M: membrane protein; N: nucleoprotein; UTR: un-translated region. Twenty-five pairs of human sera were collected 3-8 months after dose 3 and 1-3 months after dose 4 mRNA vaccine. The FFRNT50s for mNG BA.1-, BA.2-, BA.2.12.1, BA.3-, and BA.4/5-spike SARS-CoV-2s were determined in duplicate assays; the FFRNT50 for USA-WA1/2020 SARS-CoV-2 was determined in two independent experiments, each with duplicate assays. (B) FFRNT50 of sera collected before dose 4 vaccine. The bar heights and the numbers above indicate neutralizing GMTs. The whiskers indicate 95% CI. The fold of GMT reduction against each Omicron sublineage, com-pared with the GMT against USA-WA1/2020, is shown in italic font. The dotted line indicates the limit of detection of FFRNT50. FFRNT50 of <20 was treated as 10 for plot purpose and statistical analysis. The p values (Wilcoxon matched-pairs signed-rank test) for group comparison of GMTs are the following. USA-WA1/2020 versus all Omicron sublineage-spike: <0.0001; BA.1-spike versus BA.2-, BA.2.12.1-, BA.3-, BA.4/5-spike: 0.004, 0.0336, <0.0001, <0.0001, respectively; BA.2-spike versus BA.2.12.1-, BA.3-, BA.4/5-spike: 0.5, 0.065, 0.0083, respectively. BA.2.12.1-spike versus BA.3-, BA.4/5-spike: 0.0098, 0.0002, respectively; BA.3-spike versus BA.4/5-spike: 0.156. (C) FFRNT50 of sera collected after dose 4 vaccine. The p values (Wilcoxon matched-pairs signed-rank test) for group comparison of GMTs are the following. USA-WA1/2020 versus all Omicron sublineage-spike: <0.0001; BA.1-spike versus BA.2-, BA.2.12.1-, BA.3-, BA.4/5, BA.2.75-spike: 0.008, 0.033, <0.0001, <0.0001, 0.37, respectively; BA.2-spike versus BA.2.12.1-, BA.3-, BA.4/5, BA.2.75-spike: 0.12, <0.0001, <0.0001, 0.56, respectively; BA.2.12.1-spike versus BA.3-, BA.4/5, BA.2.75-spike: 0.0002, <0.0001, 0.94, respectively; BA.3-spike versus BA.4/5-, BA.2.75-spike: 0.0009, 0.13, respectively; BA.4/5-spike versus BA.2.75-spike: 0.017. (D) FFRNT50 values with connected lines for each serum pair before and after dose 4 vaccine. The GMT fold increase before and after dose 4 is shown in italic font. The p values of GMT (Wilcoxon matched-pairs signed-rank test) be-fore and after dose 4 vaccines are all <0.0001. The p values (Friedman with Dunn's multiple comparisons test) for group comparison of the increase in neutralization of sera against different variants are the following. BA.4/5-spike (5.6 folds) versus WA1 (10.8 folds), BA.1-spike (11.2 folds), BA.2-spike (9.8 folds): 0.042, 0.0007, 0.048, respectively.

FIG. 14A-14B. Neutralization of Omicron sublineages after 2 or 3 doses of mRNA vaccine and BA.1 infection. (A) FFRNT50 of 2-dose-vaccine-plus-BA.1-infection sera. Twenty-nine sera were collected from individuals who received 3 doses of vaccine and subsequently contracted BA.1 breakthrough infection. The GMT reduction fold against each Omicron sublineage and USA-WA1/2020 is shown in italic font. The dotted line indicates the limit of detection of FFRNT50. FFRNT50 of <20 was treated as 10 for plot purpose and statistical analysis. USA-WA1/2020 versus BA.1-spike, other sublineage-spikes: 0.053, <0.0001, respectively; BA. 1-spike versus other sublineage-spike: <0.0001; BA.2-spike versus BA.2.12.1-, BA.3-, BA.4/5-spike: 0.0006, 0.0215, <0.0001, respectively; BA.2.12.1-spike versus BA.3- and BA.4/5-spike: 0.0309, <0.0001, respectively; BA.3-spike versus BA.4/5-spike SARS-CoV-2: <0.0001. (B) FFRNT50 of 2-dose-vaccine-plus-BA.1-infection sera. Thirty-eight sera were collected from individuals who received 3 doses of vaccine and subsequently contracted BA.1 infection. The p values (Wilcoxon matched-pairs signed-rank test) for group comparison of GMTs are indicated below. USA-WA1/2020 versus all Omicron sublineage-spike: <0.0001; BA.1-spike versus other sublineage-spike: <0.0001; BA.2-spike versus Omicron BA.2.12.1-, BA.3-, BA.4/5, BA.2.75-spike: 0.0082, 0.9095, <0.0001, 0.093, respectively; BA.2.12.1-spike versus Omicron BA.3-, BA.4/5, BA.2.75-spike: 0.0018, <0.0001, 0.58, respectively; BA.3-spike versus BA.4/5-, BA.2.75-spike: <0.0001, 0.062, respectively; BA.4/5-spike versus BA.2.75-spike: <0.0001.

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment, and not intended to imply that the scope of the disclosure, including the claims, is limited to that embodiment.

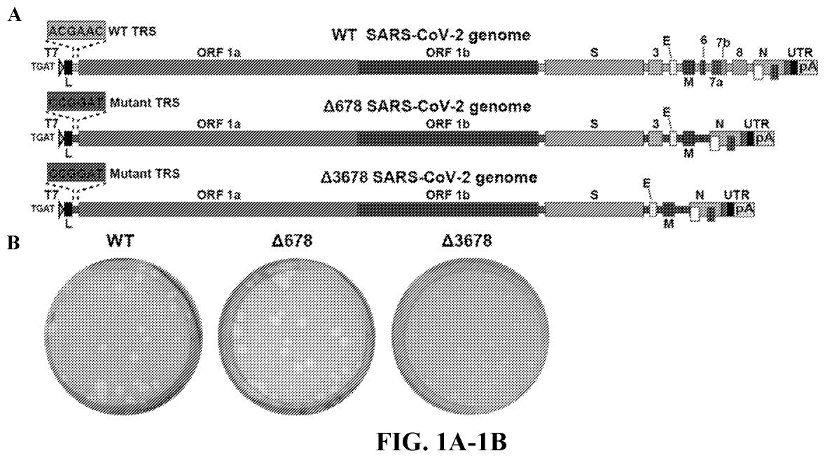

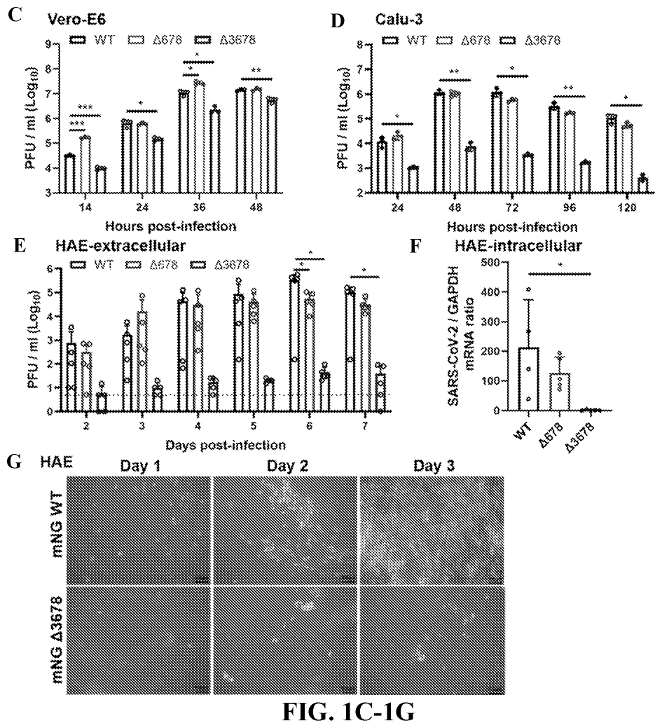

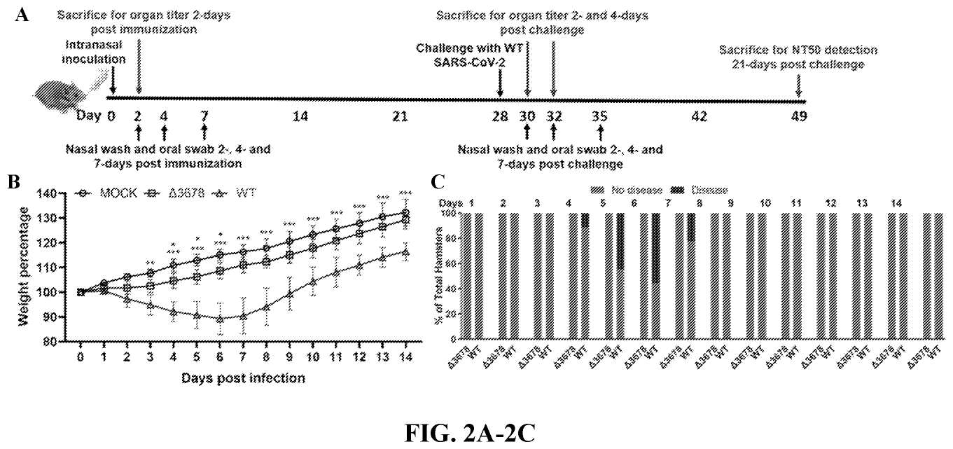

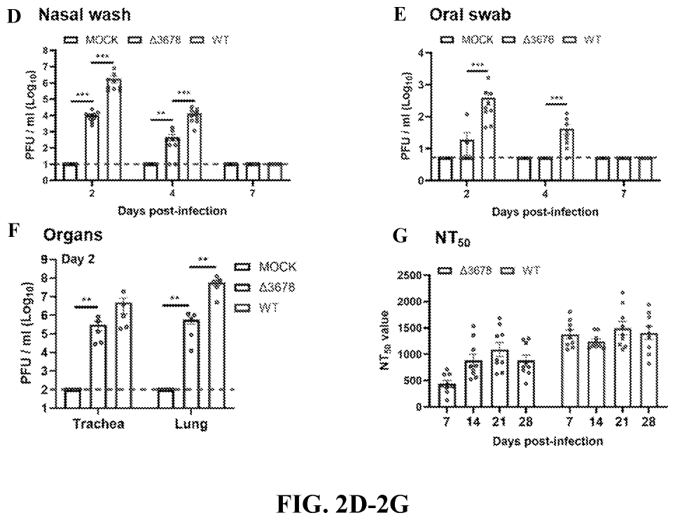

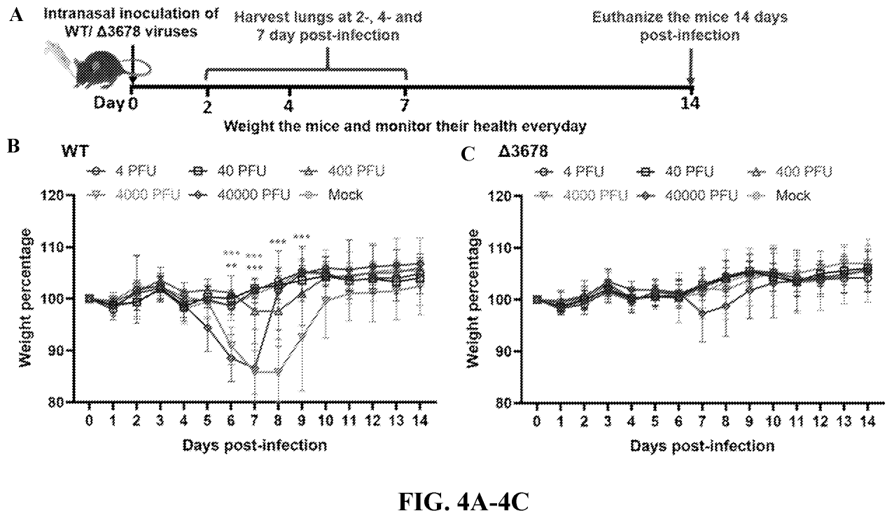

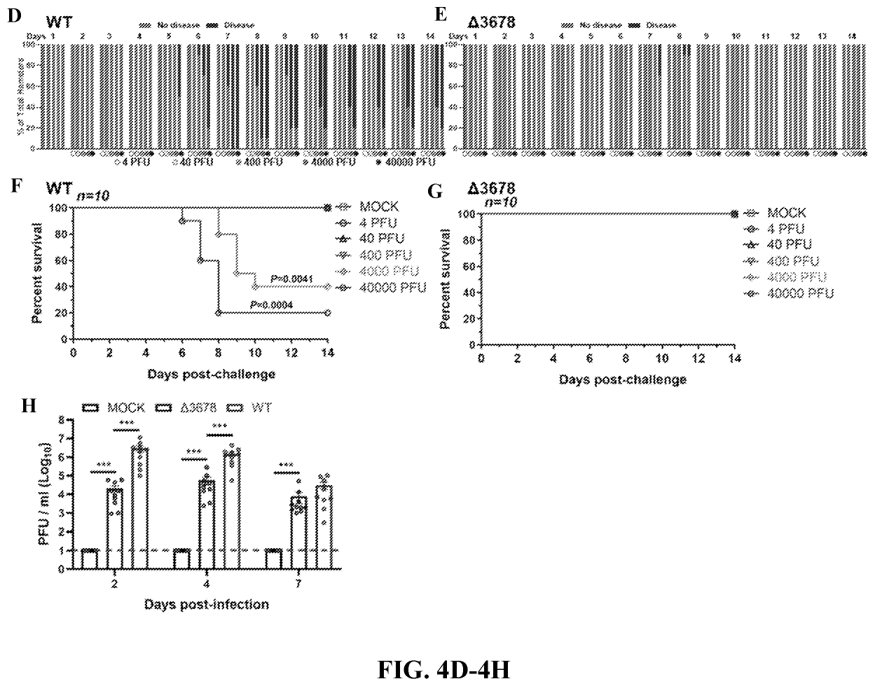

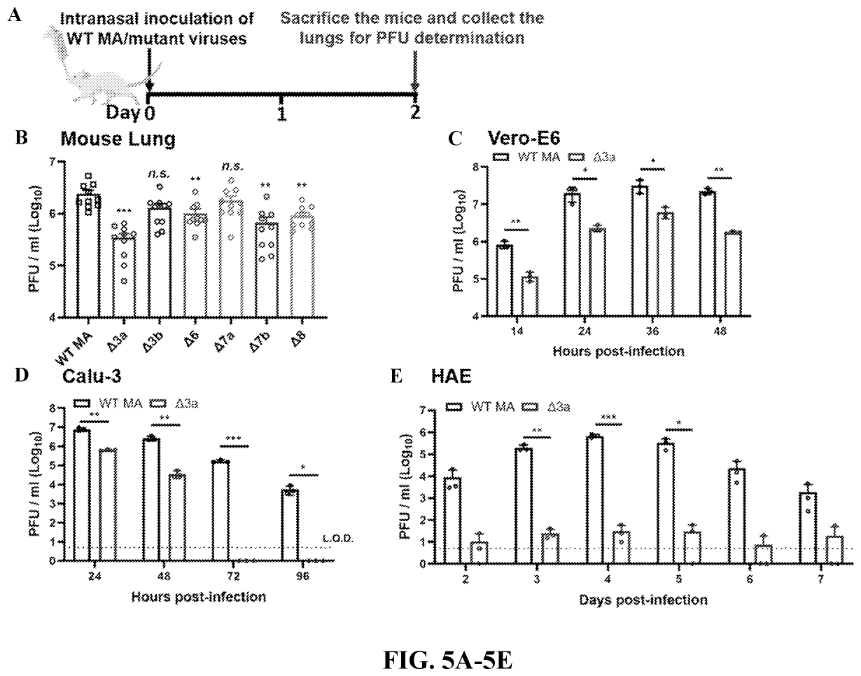

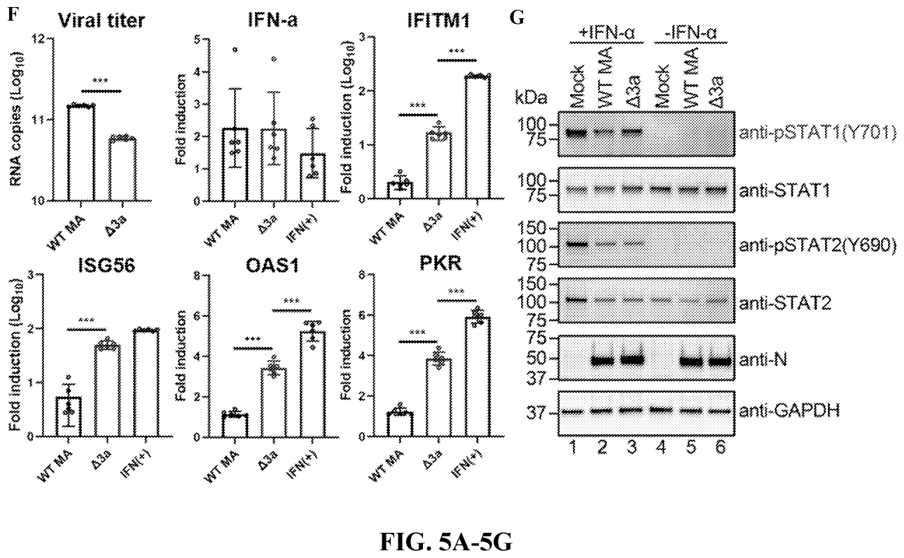

The Inventors have developed Δ3678 SARS-CoV-2 as a live-attenuated vaccine candidate. The Δ3678 virus can grow to >5.6×10⁶ PFU/ml on interferon-incompetent Vero-E6 cells (FIG. 1C), making it feasible for large-scale production. In contrast, the Δ3678 virus was highly attenuated when infecting immune-competent cells, as evidenced by the 7,500-fold reduction in viral replication than WT virus on human primary HAE cells (FIG. 1E). In both hamster and K18-hACE2 mouse models, the Δ3678 infection did not cause significant weight loss or death at the highest tested infection dose [10⁶ PFU for hamsters (FIG. 2B) and 4×10⁴ PFU for K18-hACE2 mice (FIG. 4C, 4G)], whereas the WT virus caused weight loss and death at a much lower infection dose (>4×10² PFU for K18-hACE2 mice; FIG. 4B, 4F). Analysis of individual ORF-deletion viruses identified ORF3a as a major accessory protein responsible for the attenuation of the Δ3678 virus (FIG. 5B); this conclusion was further supported by the observation that the addition of Δ3a to the Δ678 virus significantly increased the attenuation of Δ3678 replication (FIG. 1C-1G). Mechanistically, it was found that ORF3a protein antagonized innate immune response by blocking STAT1 phosphorylation during type-I interferon signaling. Thus, deletion of ORF3a conferred SARS-CoV-2 more susceptible to type-I interferon suppression. These findings have uncovered a previously uncharacterized role of ORF3a in the context of SARS-CoV-2 infection.

The attenuated Δ3678 virus may be pivoted for a veterinarian vaccine. SARS-CoV-2 can infect a variety of animal species, among which cats, ferrets, fruit bats, hamsters, minks, raccoon dogs, and white-tailed deer were reported to spread the infection to other animals of the same species (McAloose et al., mBio 11, 2020; Kuchipudi et al., Proc Natl Acad Sci USA 119(6):e2121644119, 2022; Shi et al., Science 368, 1016-20, 2020; Bonilla-Aldana et al., Vet Q 41, 250-67, 2021; Valencak et al., Geroscience 43, 2305-20, 2021). A live-attenuated Δ3678 vaccine may be useful for the prevention and control of SARS-CoV-2 on mink farms (Pomorska-Mol et al., Animal 15, 100272, 2021). Since zoonotic coronaviruses may recombine with the live-attenuated vaccine in immunized animals, the Δ3678 viruses were engineered with a mutated TRS to eliminate the possibility of recombination-mediated emergence of WT or replicative chimeric coronaviruses (FIG. 1A). This mutated TRS approach was previously shown to attenuate SARS-CoV and to prevent reversion of the WT virus (Graham et al., Commun Biol 1, 179, 2018; Yount et al., Proc Natl Acad Sci USA 103, 12546-551, 2006). Given the continuous emergence of SARS-CoV-2 variants, the vaccine antigen can be updated by swapping the variant spike glycoproteins into the current Δ3678 virus backbone.

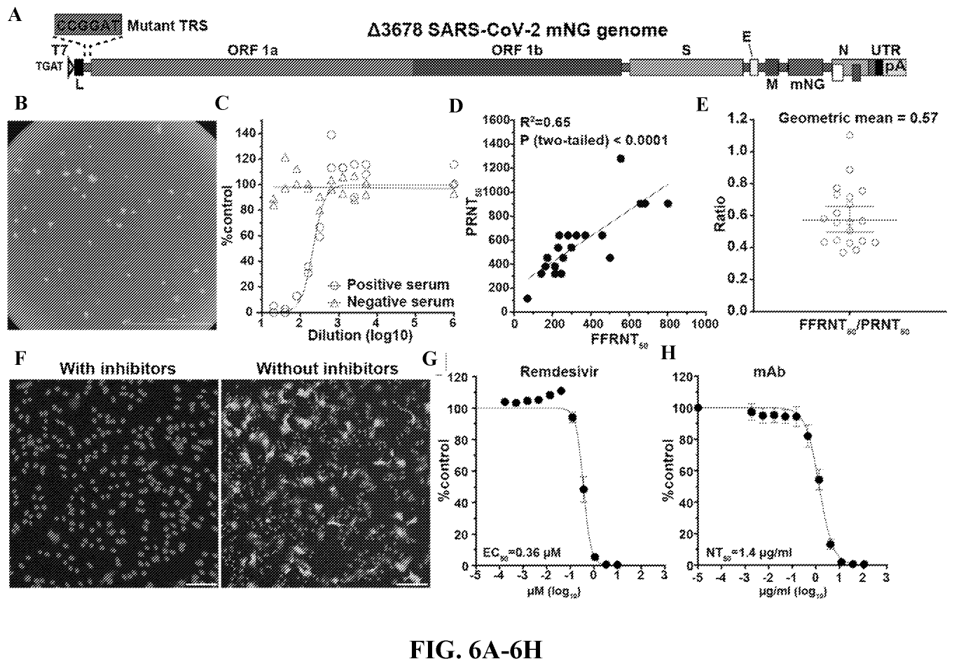

The attenuated Δ3678 virus could serve as a research tool that might be used at BSL-2. Using mNG as an example, we developed an mNG Δ3678 virus for high throughput testing of antibody neutralization and antiviral inhibitors (FIG. 6). Depending on research needs, other reporter genes, such as luciferase or other fluorescent genes, could be engineered into the system. This high-throughput assay can be modified for testing neutralization against different variants by swapping the variant spike genes into the Δ3678 backbone. The approach has been successfully used to study vaccine-elicited neutralization against variants in the context of complete SARS-CoV-2 (Liu et al., N Engl J Med 384, 1466-68, 2021; Liu et al., N Engl J Med 385, 472-74, 2021; Liu et al., Nature, 2021). Finally, in vitro and in vivo attenuation results support the use of the Δ3678 virus at BSL-2. If further attenuation is needed, more mutations, such as inactivating the NSP16 2′-O methyltransferase activity (Menachery et al., J Virol 88, 4251-64, 2014), can be rationally engineered into the Δ3678 virus.

The OFR 3b, 6, 7b, or 8 deletions reduced the lung viral loads in the K18-hACE2 mice (FIG. 5B). SARS-CoV-2 ORF8 protein was recently reported to contain a histone mimic that could disrupt chromatin regulation and enhance viral replication (Kee et al., bioRxiv, doi:10.1101/2021.1111.1110.468057, 2022). The results indicate that Δ3678 virus can serve as a live-attenuated vaccine candidate and as an experimental system that can likely be performed at BSL-2 for COVID-19 research and countermeasure development.

One utility of the described reverse genetic system described herein is to facilitate antiviral testing and therapeutic development. The reporter virus allows the use of fluorescence as a surrogate readout for viral replication. Compared with a standard plaque assay or TCID₅₀ quantification, the fluorescent readout shortens the assay turnaround time by several days. In addition, the fluorescent readout offers a quantitative measure that is less labor-intensive than the traditional means of viral titer reduction.

In certain embodiments, a kit can contain nucleic acids and/or expression vectors described herein, as well as transfection and culture reagents. A standard operating procedure (SOP) can provide guidance for use of the kit. The kit system can be used for a variety of research endeavors.

### I. CORONAVIRUSES

Coronaviruses (order Nidovirales, family Coronaviridae) are a diverse group of enveloped, positive-stranded RNA viruses. The coronavirus genome, approximately 27-32 Kb in length, is the largest found in any of the RNA viruses. Large Spike (S) glycoproteins protrude from the virus particle giving coronaviruses a distinctive corona-like appearance when visualized by electron microscopy. Coronaviruses infect a wide variety of species, including canine, feline, porcine, murine, bovine, avian and human (Holmes, et al., 1996, Coronaviridae: the viruses and their replication, p. 1075-1094, Fields Virology, Lippincott-Raven, Philadelphia, Pa.). However, the natural host range of each coronavirus strain is narrow, typically consisting of a single species. Coronaviruses typically bind to target cells through Spike-receptor interactions and enter cells by receptor mediated endocytosis or fusion with the plasma membrane (Holmes, et al., 1996, supra).

Upon entry into susceptible cells, the open reading frame (ORF) nearest the 5′ terminus of the coronavirus genome is translated into a large polyprotein. This polyprotein is autocatalytically cleaved by viral-encoded proteases, to yield multiple proteins that together serve as a virus-specific, RNA-dependent RNA polymerase (RdRP). The RdRP replicates the viral genome and generates 3′ coterminal nested subgenomic RNAs. Subgenomic RNAs include capped, polyadenylated RNAs that serve as mRNAs, and antisense subgenomic RNAs complementary to mRNAs. In one embodiment, each of the subgenomic RNA molecules shares the same short leader sequence fused to the body of each gene at conserved sequence elements known as intergenic sequences (IGS), transcriptional regulating sequences (TRS) or transcription activation sequences. It has been controversial as to whether the nested subgenomic RNAs are generated during positive or negative strand synthesis; however, recent work favors the model of discontinuous transcription during minus strand synthesis (Sawicki, et al., 1995, Adv. Exp. Med. Biol. 380:499-506: Sawicki and Sawicki Adv. Expt. Biol. 1998, 440:215).

A SARS-CoV-2 reference sequence can be found in GenBank accession NC_045512.2 as of Mar. 2, 2020. This sequence is a 29903 bp ss-RNA and is referred to as the Wuhan seafood market pneumonia virus isolate Wuhan-Hu-1. The virus is Severe acute respiratory syndrome coronavirus 2 (SARS-CoV-2) with the taxonomy of Viruses; Riboviria; Nidovirales; Cornidovirineae; Coronaviridae; Orthocoronavirinae; Betacoronavirus; Sarbecovirus. (Wu et al. “A novel coronavirus associated with a respiratory disease in Wuhan of Hubei province, China” Unpublished; NCBI Genome Project, Direct Submission, Submitted (17 Jan. 2020) National Center for Biotechnology Information, NIH, Bethesda, MD 20894, USA; Wu et al. Direct Submission, Submitted (5 Jan. 2020) Shanghai Public Health Clinical Center and School of Public Health, Fudan University, Shanghai, China).

The genome of SARS-CoV-2 referencing accession OM319525 (SEQ ID NO:10) includes (1) a 5′UTR (1-265), (2) Orflab gene (266-21555), S gene encoding a spike protein (21563 . . . 25384), ORF3a gene (25393 . . . 26220), E gene encoding E protein (26245 . . . 26472), M gene (26523 . . . 27191), ORF6 gene (27202 . . . 27387), ORF7a gene (27394 . . . 27759), ORF7b gene (27756 . . . 27887), ORF8 gene (27894 . . . 28259), N gene (28274 . . . 29533), ORF10 gene (29558 . . . 29674), and 3UTR (29675 . . . 29903). In certain aspects, ORF7 (7a and 7b) is substituted by a nucleic acid encoding a reporter protein. Corresponding regions and segment of SEQ ID NO:1 can be determined by alignment with the OM319525 (SEQ ID NO:10) sequence or other similar coronaviruses.

The reporter protein is a protein that can be detected, directly or indirectly, and includes colorimetric, fluorescent or luminescent proteins, as well as proteins that bind affinity reagents such as protein/ligand pairs and protein/antibody pairs. Examples of luminescent or marker proteins that can be used in embodiments of the invention include, but are not limited to, Aequorin, firefly luciferase, Renilla luciferase, red luciferase, luxAB, and nanoluciferase. Examples of chemiluminescent protein or marker protein include 0-galactosidase, horseradish peroxidase (HRP), and alkaline phosphatase. Examples of fluorescent protein or marker protein include, but are not limited to, mNeonGreen, TagBFP, Azurite, EBFP2, mKalama1, Sirius, Sapphire, T-Sapphire, ECFP, Cerulean, SCFP3A, mTurquoise, monomeric Midoriishi-Cyan, TagCFP, mTFP1, EGFP, Emerald, Superfolder GFP, Monomeric Azami Green, TagGFP2, mUKG, mWasabi, EYFP, Citrine, Venus, SYFP2, TagYFP, Monomeric Kusabira-Orange, mKOK, mKO2, mOrange, mOrange2, mRaspberry, mCherry, dsRed, mStrawberry, mTangerine, tdTomato, TagRFP, TagRFP-T, mApple, mRuby, mPlum, HcRed-Tandem, mKate2, mNeptune, NirFP, TagRFP657, IFP1.4, iRFP, mKeima Red, LSS-mKatel, LSS-mKate2, PA-GFP, PAmCherryl, PATagRFP, Kaede (green), Kaede (red), KikGR1 (green), KikGR1 (red), PS-CFP2, PS-CFP2, mEos2 (green), mEos2 (red), PSmOrange, or Dronpa.

### II. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### A. Results

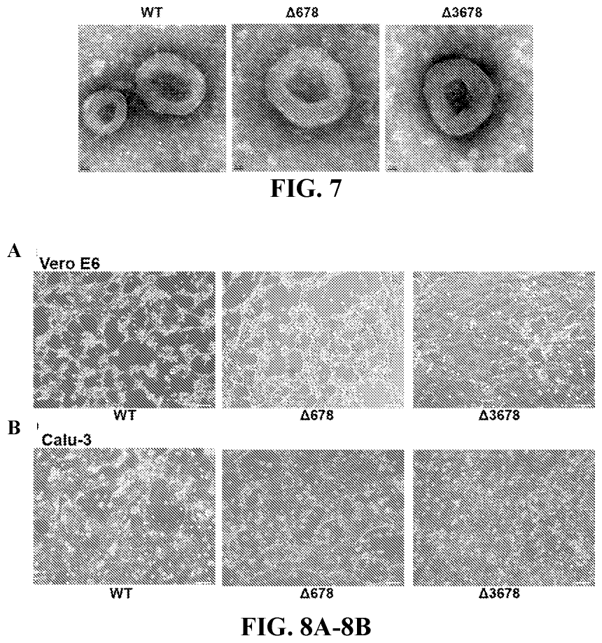

Attenuation of SARS-CoV-2 by deletion of accessory genes. Using an infection clone of USA-WA1/2020 SARS-CoV-2, two mutant viruses were constructed containing accessory ORF deletions (FIG. 1a), one with ORF 6, 7, and 8 deletions (Δ678, SEQ ID NO:3) and another with ORF 3, 6, 7, and 8 deletions (Δ3678, SEQ ID NO:1). Besides the ORF deletions, the viral transcription regulatory sequences (TRS) of both Δ678 and Δ3678 viruses were mutated from the wild-type (WT) ACGAAC to CCGAT (mutant nucleotides underlined; FIG. 1a). The mutated TRS eliminates the possibility to produce WT SARS-CoV-2 through recombination between the Δ678 or Δ3678 RNA and inadvertently contaminating viral RNA^{21,22}. On Vero-E6 cells, the Δ678 virus developed plaques similar to the WT virus, whereas the Δ3678 virus produced smaller plaques (FIG. 1b). Both Δ678 and Δ3678 viruses were visible under the negative staining electron microscope (FIG. 7). Replication kinetics analysis showed that WT and Δ678 replicated to comparable viral titers on Vero-E6 (FIG. 1c), Calu-3 (FIG. 1d), and primary human airway epithelial (HAE) cultures (FIG. 1e). In contrast, the replication of Δ3678 was slightly attenuated on Vero-E6 cells (FIG. 1c), but became significantly more attenuated on Calu-3 (360-fold lower peak viral titer than WT virus at 72 h; FIG. 1d) and HAE culture (7,500-fold lower peak viral titer than WT virus on day 6; FIG. 1e). Consistently, the intracellular level of Δ3678 RNA was about 100-fold lower than that of Δ678 RNA and WT in HAE cells (FIG. 1f). Corroboratively, the Δ3678 virus caused much less cytopathic effects (CPE) than the Δ678 and WT viruses on both Vero-E6 and Calu-3 cells (FIG. 8). To further confirm the attenuation of Δ3678 virus, the mNeonGreen (mNG) gene was engineered into the Δ3678 (SEQ ID NO:2) and WT viruses²³. When infecting the HAE culture, the mNG Δ3678 virus developed significantly fewer mNG-positive cells than the mNG WT virus (FIG. 1g). Taken together, the results indicate that (i) deletions of ORFs 6, 7, and 8 hardly attenuate SARS-CoV-2 in cell culture; (ii) an additional deletion of ORF3 to the Δ678 virus significantly increases the attenuation of Δ3678; and (iii) the Δ3678 virus is strikingly more attenuated when infecting immune-competent cells than when infecting interferon-deficient cells.

Characterization of Δ3678 SARS-CoV-2 as a potential live-attenuated vaccine in a hamster model. The attenuation of Δ3678 virus was characterized in a hamster model (FIG. 2a). After intranasal infection with 10⁶ plaque-forming units (PFU) of Δ3678, the hamsters did not lose weight (FIG. 2b) or develop disease (FIG. 2c), whereas the WT virus-infected animals lost weight (FIG. 2b) and developed disease (FIG. 2c). On day 2 post-infection, viral loads in the Δ3678-infected nasal wash (FIG. 2d), oral swab (FIG. 2e), trachea, and lung (FIG. 2f) were 180-, 20-, 16-, and 100-fold lower than those in the WT-infected specimens. The Δ3678 infection elicited robust neutralization with a peak 50% neutralization titer (NT₅₀) of 1,090 on day 21 post-infection, while the WT virus evoked 1.4-fold higher peak NT₅₀ (FIG. 2g). The results demonstrate that the Δ3678 virus is attenuated and can elicit robust neutralization in hamsters.

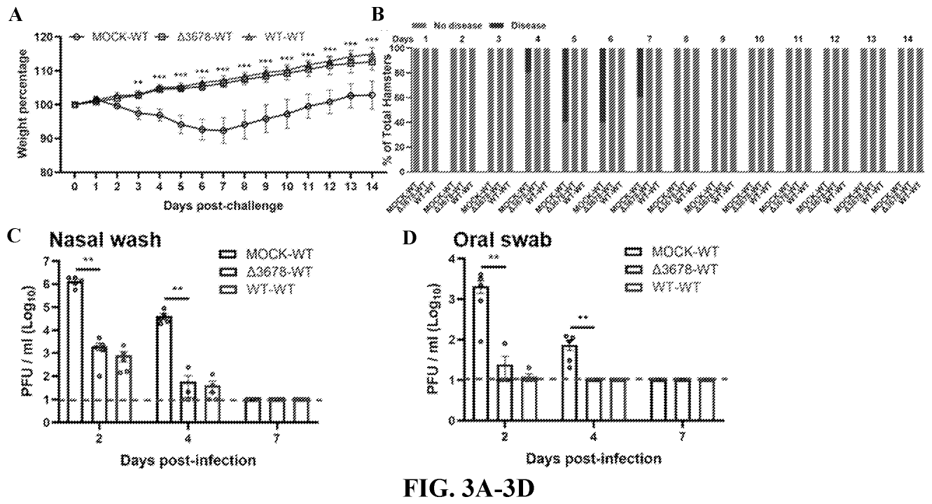

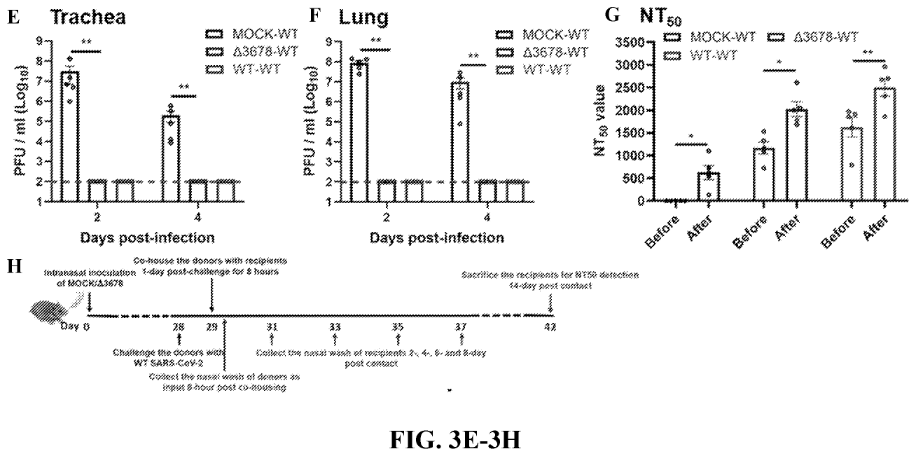

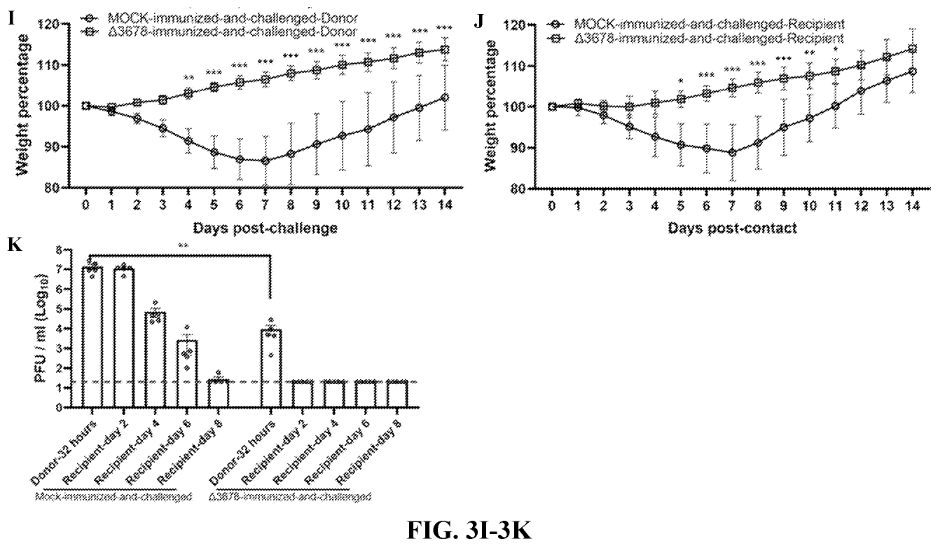

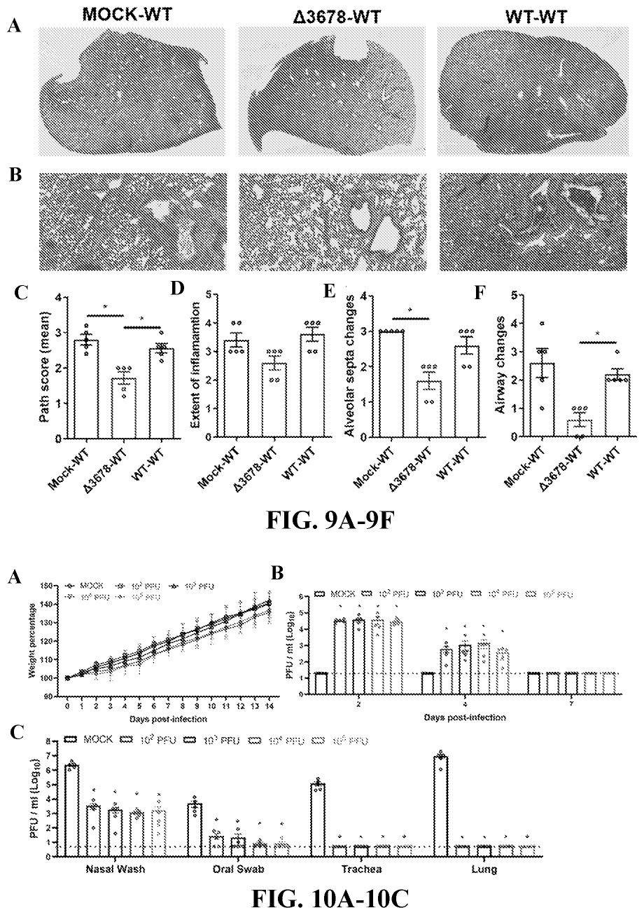

Whether the above immunized hamsters could be protected from SARS-CoV-2 challenge was examined. After intranasal challenge with 10⁵ PFU of WT SARS-CoV-2 on day 28 post-immunization (FIG. 2a), both the Δ3678- or WT virus-immunized animals were protected from weight loss (FIG. 3a) or disease (FIG. 3b). Compared with the mock-immunized group, the viral loads in the nasal wash (FIG. 3c) and oral swab (FIG. 3d) from the Δ3678- and WT virus-immunized groups were decreased by >660 (day 2) and >80 folds (day 2), respectively; no infectious viruses were detected in trachea (FIG. 3e) and lungs (FIG. 3f) from the immunized groups. The challenge significantly increased the neutralization titers (on day 21 post-challenge) in both the Δ3678- and WT virus-immunized groups (FIG. 3g), suggesting that a single infection with the Δ3678 or WT virus did not evoke sterilizing immunity. Histopathology analysis showed that immunization with attenuated Δ3678 virus reduced lung pathology score, inflammation, alveolar septa change, and airway damage (FIG. 9). In contrast, the previous infection with WT virus did not exhibit improved lung histopathology after the challenge, possibly because the observed pathology was caused by the initial WT viral infection (FIG. 9). Collectively, the results demonstrate that immunization with attenuated Δ3678 virus can protect WT SARS-CoV-2 challenge in hamsters.

Next, lower dose immunization was tested to see if it could also achieve protection. Hamsters were immunized with 10², 10³, 10⁴, or 10⁵ PFU of Δ3678 virus. No weight loss (FIG. 10a) and equivalently reduced lung viral loads (FIG. 10b) were observed for all dose groups. After challenge with WT SARS-CoV-2, all dose groups exhibited protection similar to the 10⁶-PFU-dose group, including no viral loads in the trachea or lung and significantly reduced viral loads in the nasal wash and oral swab (FIG. 10c). The results indicate a low dose of 10² PFU of Δ3678 immunization is protective in hamsters.

Since infectious viruses were detected in the nasal and oral specimens from the Δ3678-immunized hamsters after the challenge, we examined whether such low levels of virus could be transmitted to naïve hamsters. On day 1 post-challenge, the Δ3678-immunized-and-challenged animals (donor) were co-housed with clean naïve hamsters (recipient) for 8 h, after which the donor and recipient animals were separated (FIG. 3h). As expected, after WT virus challenge, no weight loss was observed in the Δ3678-immunized donor animals, but not in the mock-immunized donor animals (FIG. 3i). After co-housing with Δ3678-immunized-and-challenged donor animals, recipient animals did not lose weight (FIG. 3j) and did not have infectious viruses in the nasal wash (FIG. 3k). In contrast, after co-housing with the mock-immunized-and-challenged donor animals, the recipient animals lost weight (FIG. 3j) and developed high viral loads in the nasal wash (FIG. 3k). Altogether, the results indicate that although the Δ3678-immunized donor animals developed low viral loads in their nasal and oral specimens after the WT virus challenge, they might not be able to transmit the virus to clean naïve hamsters.

Attenuation of Δ3678 SARS-CoV-2 in K18-hACE2 mice. To further characterize the attenuation of Δ3678, K18-hACE2 mice were intranasally inoculated with 4, 40, 400, 4,000, or 40,000 PFU of WT or Δ3678 virus (FIG. 4a). The infected mice were compared for their weight loss, survival rates, and disease symptoms between the WT and Δ3678 groups. The WT viral infection caused weight loss at doses of >400 PFU (FIG. 4b), diseases at dose of >400 PFU (FIG. 4d), and deaths at doses of >4,000 PFU (FIG. 4f). In contrast, the Δ3678 virus caused slight (statistically insignificant) weight loss at 40,000 PFU (FIG. 4c), transient diseases at 4,000 PFU (FIG. 4e), and no death at all doses (FIG. 4g). Consistently, the lung viral loads from the Δ3678-infected mice were significantly lower than those from the WT-infected animals (FIG. 4h). The results demonstrated that Δ3678 virus was highly attenuated in the K18-hACE2 mice.

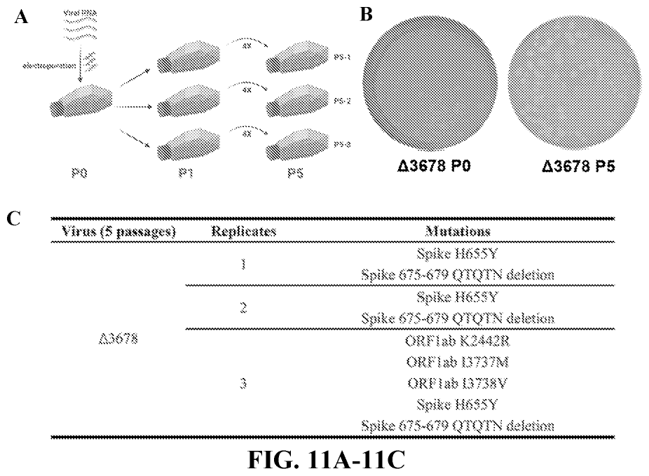

Genetic stability of Δ3678 SARS-CoV-2 on Vero-E6 cells. Given the potential of Δ3678 as a live-attenuated vaccine, we examined its genetic stability by continuously culturing the virus for five rounds on Vero-E6 cells. Three independent passaging experiments were performed to assess the consistency of adaptive mutations (FIG. 11a). The passage 5 (P5) virus developed bigger plaques than the original P0 virus (FIG. 11b). Full-genome sequencing of the P5 viruses identified H655Y mutation and 675-679 QTQTN deletion from all three selections (FIG. 11c). The mutation and deletion are located immediately upstream of the furin cleavage site between the spike 1 and 2 subunits. Previous studies showed that culturing of SRAS-CoV-2 on Vero cells expressing serine protease TMPRSS2 could eliminate such mutation/deletion (Lau et al., Emerg Microbes Infect 9, 837-42, 2020; Chen et al., Nat Med 27, 717-26, 2021; Johnson et al., Nature 591, 293-99, 2021).

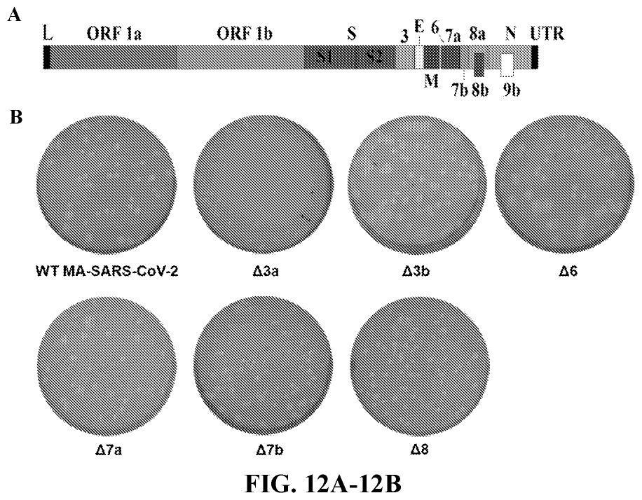

Contribution of individual ORFs to the attenuation of Δ3678 virus. To define the role of each ORF in attenuating Δ3678 virus, a panel of mutant viruses were prepared in the backbone of a mouse-adapted SARS-CoV-2 (MA-SARS-CoV-2) that can robustly infect BALB/c mice (Muruato et al., PLoS Biol 19, e3001284, 2021). Each mutant virus contained a single accessory gene deletion, including Δ3a, Δ3b, Δ6, Δ7a, Δ7b, or Δ8. Among all the individual deletion mutants, Δ3a virus developed the smallest plaques on Vero-E6 cells (FIG. 12). The biological importance of each deleted gene was analyzed by viral replication in the lungs after intranasal infection of BALB/c mice (FIG. 5a). On day 2 post-infection, deletion of Δ3a, Δ3b, Δ6, Δ7b, or Δ8 reduced viral loads in lungs, among which Δ3a exhibited the largest reduction (FIG. 5b). To further confirm the critical role of Δ3a in viral attenuation, the replication kinetics of Δ3a and WT MA-SARS-CoV-2 were compared on Vero-E6 (FIG. 5c), Calu-3 (FIG. 5d), and HAE cultures (FIG. 5e). The replication of Δ3a was significantly more attenuated on immune-competent Calu-3 and HAE cells than that on interferon-deficient Vero-E6 cell (FIG. 5c-5e). Taken together, the results indicate that Δ3a played a major role in attenuating the Δ3678 virus, possibly through the type-I interferon pathway.

ORF3a antagonizes type-I interferon signaling through inhibiting STAT1 phosphorylation. To define the mechanism of Δ3a-mediated viral attenuation, A549 cells expressing human ACE2 receptor (A549-hACE2) were infected with Δ3a or WT MA-SARS-CoV-2. Although the replication of Δ3a was lower than the WT virus, comparable levels of IFN-α RNA were produced (FIG. 5f). Significantly higher levels of interferon-stimulating genes (ISGs), such as IFITM1, ISG56, OAS1, and PKR, were detected in the Δ3a virus-infected cells than those in the WT virus-infected cells (FIG. 5f), suggesting a role of ORF3a in suppressing type-I interferon signaling. To further support this conclusion, the A549-hACE cells were treated with IFN-α followed by Δ3a or WT virus infection. Western blot analysis showed that the phosphorylation of STAT1 was less efficient in the Δ3a-infected cells than the WT-infected cells, whereas no difference in STAT2 phosphorylation was observed (FIG. 5g). Thus, the results indicate that ORF3a protein suppresses STAT1 phosphorylation during type-I interferon signaling.

An mNG reporter Δ3678 virus for neutralization and antiviral testing. The in vitro and in vivo attenuation results suggest that Δ3678 virus may serve as a research tool for BSL-2 use. To further develop this tool, an mNG gene (driven by its own TRS sequence) was engineered between the M and N genes of the Δ3678 genome, resulting in mNG Δ3678 virus (FIG. 6a, SEQ ID NO:2). For high-throughput neutralization testing, the mNG Δ3678 virus was developed into a fluorescent focus reduction neutralization test (FFRNT) in a 96-well format. When infecting Vero-E6 cells, the mNG Δ3678 developed fluorescent foci that could be quantified by high content imaging (FIG. 6b). FIG. 6c shows the FFRNT curves for one COVID-19 convalescent positive serum one negative serum. To validate the FFRNT assay, 20 convalescent sera were tested against the mNG Δ3678 virus. For comparison, the same serum panel was tested against the WT SARS-CoV-2 (without mNG) using the gold-standard plaque-reduction neutralization test (PRNT) (Muruato et al., Nat Commun 11, 4059, 2020). The 50% reduction neutralization titers (NT₅₀) correlated well between the FFRNT and PRNT assays (FIG. 6d). The geometric mean of FFRNT₅₀/PRNT₅₀ ratio is 0.57 for the tested serum panel (FIG. 6e). Next, the reporter mNG Δ3678 virus was developed into a high-throughput antiviral assay. Treatment of the mNG Δ3678 virus-infected A549-hACE2 cells with remdesivir diminished the appearance of mNG-positive cells (FIG. 6f). Dose-responsive antiviral curves were reliably obtained for small molecule remdesivir (FIG. 6g) and for a monoclonal antibody (FIG. 6h). Overall, the results demonstrate that mNG Δ3678 virus could be used for high-throughput neutralization and antiviral testing.

### B. Methods

Ethics statement. Hamster and mouse studies were performed in accordance with the guidance for the Care and Use of Laboratory Animals of the University of Texas Medical Branch (UTMB). The protocol was approved by the Institutional Animal Care and Use Committee (IACUC) at UTMB. All the animal operations were performed under anesthesia by isoflurane to minimize animal suffering. The use of human COVID-19 sera was reviewed and approved by the UTMB Institutional Review Board (IRB #: 20-0070). The convalescent sera from COVID-19 patients (confirmed by the molecular tests with FDA's Emergency Use Authorization) were leftover specimens and completely de-identified from patient information. The serum specimens were heat-inactivated at 56° C. for 30 min before testing.

Animals and Cells. The Syrian golden hamsters (HsdHan:AURA strain) were purchased from Envigo (Indianapolis, IN). K18-hACE2 mice were purchased from the Jackson Laboratory (Bar Harbor, ME). BALB/c mice were purchased from Charles River Laboratories (Wilmington, MA). African green monkey kidney epithelial Vero-E6 cells (laboratory-passaged derivatives from ATCC CRL-1586) were grown in Dulbecco's modified Eagle's medium (DMEM; Gibco/Thermo Fisher, Waltham, MA, USA) with 10% fetal bovine serum (FBS; HyClone Laboratories, South Logan, UT) and 1% antibiotic/streptomycin (P/S, Gibco). Vero-E6-TMPRSS2 cells were purchased from SEKISUI XenoTech, LLC (Kansas City, KS) and maintained in 10% fetal bovine serum (FBS; HyClone Laboratories, South Logan, UT) and 1% P/S and 1 mg/ml G418 (Gibco). The A549-hACE2 cells that stably express hACE2 were grown in the DMEM supplemented with 10% fetal bovine serum, 1% P/S and 1% 4-(2-hydroxyethyl)-1-piperazineethanesulfonic acid (HEPES); ThermoFisher Scientific) and 10 μg/mL Blasticidin S. Human lung adenocarcinoma epithelial Calu-3 cells (ATCC, Manassas, VA, USA) were maintained in a high-glucose DMEM containing sodium pyruvate and GlutaMAX (Gibco) with 10% FBS and 1% penicillin/streptomycin at 37° C. with 5% CO₂. The EpiAirway system is a primary human airway 3D tissue model purchased from MatTek Life Science (Ashland, MA, USA). All cells were maintained at 37° C. with 5% CO₂. All cell lines were verified and tested negative for mycoplasma.

Generation of SARS-CoV-2 mutant viruses. (1) Generate mutant viruses with accessory ORF deletions. The ORF 6, 7, and 8 deletions (Δ678) and ORF 3, 6, 7, and 8 deletions (Δ3678) were constructed by overlap PCR using an infection clone of USA-WA1/2020 SARS-CoV-2 (Xie et al., Cell Host Microbe 27, 841-48 e843, 2020). The Δ3a, Δ3b, Δ6, Δ7a, Δ7b, Δ8 mutants were constructed by overlap PCR using an infection clone of a mouse-adapted SARS-CoV-2 (MA-SARS-CoV-2) (Muruato et al., PLoS Biol 19, e3001284, 2021). (2) Generate reporter viruses with accessory ORF deletions. The mNG WT and mNG Δ3678 SARS-CoV-2s were generated by engineering the mNeonGreen (mNG) gene into the ORF7 position of the WT and Δ3678 viruses. The mutant infectious clones were assembled by in vitro ligation of contiguous DNA fragments following the protocol previously described (Xie et al., Nature Protocols 16, 1761-84, 2021). In vitro transcription was then performed to synthesize genomic RNA. For recovering the viruses, the RNA transcripts were electroporated into Vero-E6 cells. The viruses from electroporated cells were harvested at 40 h post-electroporation and served as P0 stocks. All viruses were passaged once on Vero-E6 cells for subsequent experiments and sequenced after RNA extraction to confirm no undesired mutations. Viral titers were determined by plaque assay on Vero-E6 cells. All virus preparation and experiments were performed in a BSL-3 facility. Viruses and plasmids are available from the World Reference Center for Emerging Viruses and Arboviruses (WRCEVA) at the University of Texas Medical Branch.

RNA extraction, RT-PCR, and cDNA sequencing. Cell culture supernatants or clarified tissue homogenates were mixed with a five-fold excess of TRIzol™ LS Reagent (Thermo Fisher Scientific, Waltham, MA). Viral RNAs were extracted according to the manufacturer's instructions. The extracted RNAs were dissolved in 20 μl nuclease-free water. For sequence validation of mutant viruses, 2 μl of RNA samples were used for reverse transcription by using the SuperScript™ IV First-Strand Synthesis System (Thermo Fisher Scientific) with random hexamer primers. Nine DNA fragments flanking the entire viral genome were amplified by PCR. The resulting DNAs were cleaned up by the QIAquick PCR Purification Kit, and the genome sequences were determined by Sanger sequencing at GENEWIZ (South Plainfield, NJ).

Viral infection of cell lines. Approximately 3×10⁵ Vero-E6 or Calu-3 cells were seeded onto each well of 12-well plates and cultured at 37° C., 5% CO₂ for 16 h. SARS-CoV-2 WT or mutant viruses were inoculated onto Vero-E6 and Calu-3 cells at an MOI of 0.01 and 0.1, respectively. After 2 h infection at 37° C. with 5% CO₂, the cells were washed with DPBS 3 times to remove any detached virus. One milliliter of culture medium was added to each well for the maintenance of the cells. At each time point, 100 μl of culture supernatants were collected for detection of virus titer, and 100 μl of fresh medium was added into each well to replenish the culture volume. The cells were infected in triplicate for each group of viruses. All samples were stored at −80° C. until analysis.

Viral infection in a primary human airway cell culture model. The EpiAirway system is a primary human airway 3D mucociliary tissue model consisting of normal, human-derived tracheal/bronchial epithelial cells. For viral replication kinetics, WT or mutant viruses were inoculated onto the culture at an indicated MOI in DPBS. After 2 h infection at 37° C. with 5% CO₂, the inoculum was removed, and the culture was washed three times with DPBS. The infected epithelial cells were maintained without any medium in the apical well, and the medium was provided to the culture through the basal well. The infected cells were incubated at 37° C., 5% CO₂. From 1-7 days, 300 μl of DPBS were added onto the apical side of the airway culture and incubated at 37° C. for 30 min to elute the released viruses. All virus samples in DPBS were stored at −80° C.

Quantitative real-time RT-PCR assay. RNA copies of SARS-CoV-2 samples were detected by quantitative real-time RT-PCR (RT-qPCR) assays were performed using the iTaq SYBR Green One-Step Kit (Bio-Rad) on the LightCycler 480 system (Roche, Indianapolis, IN) following the manufacturer's protocols. The absolute quantification of viral RNA was determined by a standard curve method using an RNA standard (in vitro transcribed 3,480 bp containing genomic nucleotide positions 26,044 to 29,883 of SARS-CoV-2 genome).

Hamster immunization and challenge assay. Four- to six-week-old male golden Syrian hamsters, strain HsdHan:AURA (Envigo, Indianapolis, IN), were immunized intranasally with 100 μl WT virus (10⁶ PFU, n=20) or Δ3678 mutant virus (10⁶ PFU, n=20). Animals received DMEM media (supplemented with 2% FBS and 1% penicillin/streptomycin) served as Mock group (n=20). On day 28, the animals were challenged with 10⁵ PFU of WT SARS-CoV-2. The animals were weighed and monitored for signs of illness daily. Nasal washes and oral swabs were collected in 400 μl sterile DPBS and 1 ml DMEM media at indicated time points. For organ collection, animals were humanely euthanized on days 2, 30, and 32, tracheae and lungs were harvested and placed in a 2-ml homogenizer tube containing 1 ml of DMEM media. On day 49, animals were humanely euthanized for blood collection, serum were then isolated for neutralization titer (NT₅₀) detection. The NT₅₀ values were determined using an mNG USA-WA 1/2020 SARS-CoV-2 as previously reported (Muruato et al., Nat Commun 11, 4059, 2020).

To test if lower dose immunization could achieve protection, hamsters were immunized with 10², 10³, 10⁴, or 10⁵ PFU of Δ3678 virus. Nasal washes, oral swabs and organs were collected at indicated time points. The animals were weighed and monitored for signs of illness daily.

Hamster transmission assay. Hamster transmission assay was performed per our previous protocol (Liu et al., Nature, 2021). Briefly, hamsters were immunized intranasally with 10⁶ PFU Δ3678 mutant virus (n=5). Animals who received DMEM media served as a mock group (n=5). On day 28 post-immunization, the animals were challenged with 10⁵ PFU of WT SARS-CoV-2. One day later, one infected donor animal was co-housed with one naïve animal for 8 h (5 pairs for mock group, 5 pairs for Δ3678 group). After the 8-h contact, the donor and recipient animals were separated and maintained in individual cages. Nasal washes were collected at indicated time points. On day 42, animals were humanely euthanized for blood collection.

Mouse infection of Δ3678 virus. Eight- to 10-week-old K18-hACE2 female mice were intranasally infected with 50 μl different doses of WT or Δ3678 virus (4, 40, 400, 4,000, 40,000 PFU, n=10 per dose). Animals received DMEM media served as a mock group. Lungs were collected on days 2, 4, and 7 post-infection. Animals were weighed and monitored for signs of illness daily and were sacrificed on day 14.

To define the role of each ORF in attenuating Δ3678 virus, 8- to 10-week-old BALB/c female mice were intranasally infected with 50 μl WT mouse-adapted-SARS-CoV-2 (10⁴ PFU, n=10) or Δ3a, Δ3b, Δ6, Δ7a, Δ7b, Δ8 virus (10⁴ PFU, n=10 per virus). On day 2 post-infection, animals were humanely euthanized for lung collection.

Histopathology. Hamsters were euthanized with ketamine/xylazine injection and necropsy was performed. The lungs were inspected for gross lesions and representative portions of the lungs were collected in 10% buffered formalin for histology. Formalin-fixed tissues were processed per a standard protocol, 4 μm-thick sections were cut and stained with hematoxylin and eosin (HE). The slides were imaged in a digital scanner (Leica Aperio LV1). Lung sections were examined under light microscopy using an Olympus CX43 microscope for the extent of inflammation, size of inflammatory foci, and changes in alveoli, alveolar septa, airways, and blood vessels. The blinded tissue sections were semi-quantitatively scored for pathological lesions.

TABLE 1
Criteria for histopathology scoring

| | Scores Location | 0 | 1 | 2 | 3 | 4 |
|---|---|---|---|---|---|---|
| A | Extent of | 0 | <10 | 10-30 | 30-60 | >60 |
| | inflammation (% | | | | | |
| | tissue involved) | | | | | |
| B | Inflammatory | No | Patchy | Patchy | Large | Large |
| | foci type | inflammation | inflammatory | inflammatory | inflammatory | inflammatory |
| | | | foci, few | foci, many | foci, few | foci, many |
| | | | (<2) | (>2) | (<2) | (>2) |
| C | Alveolar septa | Thin and | Thickened in | Thickened in | Thickened in | Thickened in |
| | | delicate | <10% HPF | <30% HPF | <60% HPF | >60% HPF |
| D | Airways | Clear; | Few cells | Moderate cells | More cells | Occlusion of air |
| | | no cells | in airway | in airway | in air way; | way/epithelial |
| | | | | | Epithelial | hyperplasia or |
| | | | | | hyperplasia | desquamation |
| E | Alveoli/perivascular | Clear; no | Few cells. | Moderate cells/PVC/ | More cells/PVC/ | Abundant cells/ |
| | cuff/blood vessels/ | inflammatory | Few PMN | mild congestion/ | more congestion | large PVG/severe |
| | pleuritis/cell types | cells | or MNC | mild pleuritis/ | and pleuritis/more | congestion or |
| | | | | mostly MNC | MNC and PMN | pleuritis/mixed cells |

Plaque assay. Approximately 1.2×10⁶ Vero-E6 cells were seeded to each well of 6-well plates and cultured at 37° C., 5% CO₂ for 16 h. Virus was serially diluted in DMEM with 2% FBS and 200 μl diluted viruses were transferred onto the monolayers. The viruses were incubated with the cells at 37° C. with 5% CO₂ for 1 h. After the incubation, overlay medium was added to the infected cells per well. The overlay medium contained DMEM with 2% FBS, 1% penicillin/streptomycin, and 1% sea-plaque agarose (Lonza, Walkersville, MD). After a 2-day incubation, the plates were stained with neutral red (Sigma-Aldrich, St. Louis, MO) and plaques were counted on a lightbox. The detection limit of the plaque assay was 10 PFU/ml.

Genetic stability of Δ3678 SARS-CoV-2. The P0 Δ3678 SARS-CoV-2 was continuously cultured for five rounds on Vero-E6 cells. Three independent passaging experiments were performed to assess the consistency of adaptive mutations. The P5 viral RNAs from three independent replicates were extracted and subjected to RT-PCR. Whole-genome sequencing was performed on RT-PCR products. The mutations that occurred in the P5 Δ3678 viruses were analyzed.

ORF3a-mediated suppression of type-I interferon signaling. A549-hACE2 cells were infected with WT or ΔORF3a SARS-CoV-2 at an MOI of 1 for 1 h, after which the cells were washed twice with PBS and cultured in a fresh medium. Intracellular RNAs were harvested at 24 h post-infection. Viral RNA copies and mRNA levels of IFN-α, IFITM1, ISG56, OAS1, PKR, and GAPDH were determined by quantitative RT-PCR. The housekeeping gene GAPDH was used to normalize mRNA levels and the mRNA levels are presented as fold induction over mock samples. As a positive control, uninfected cells were treated with 1,000 units/ml IFN-α for 24 h.

Suppression of STAT phosphorylation by ORF3a protein. A549-hACE2 cells were pre-treated with 1,000 units/ml IFN-α for 6 h. Mock-treated cells were used as a control. Cells were infected with WT or ΔORF3 SARS-CoV-2 at an MOI 1 for 1 h. Inoculums were removed; cells were washed twice with PBS; fresh media with or without 1,000 units/ml IFN-α were added. Samples were collected at 24 h post-infection by using 2× Laemmli buffer (BioRad, #1610737) and analyzed by Western blot. Recombinant human a-interferon (1F007) was purchased from Millipore (Darmstadt, Germany). Anti-STAT1 (14994S, 1:1,000), anti-pSTAT1 (Y701) (7649S, 1:1,000), anti-STAT2 (72604S, 1:1,000), anti-pSTAT2 (Y690) (88410S, 1:1,000) antibodies were from Cell Signaling Technology (Danvers, MA); anti-GAPDH (G9545, 1:1,000) antibodies were from Sigma-Aldrich; SARS-CoV-2 (COVID-19) nucleocapsid antibody (NB100-56576, 1:1000) were from Novus Biologicals (CO, USA).

Fluorescent focus reduction neutralization test (FFRNT). Neutralization titers of COVID-19 convalescent sera were measured by a fluorescent focus reduction neutralization test (FFRNT) using mNG Δ3678 SARS-CoV-2. Briefly, Vero-E6 cells (2.5×10⁴) were seeded in each well of black μCLEAR flat-bottom 96-well plate (Greiner Bio-one™). The cells were incubated overnight at 37° C. with 5% CO₂. On the following day, each serum was 2-fold serially diluted in the culture medium with the first dilution of 1:20. Each serum was tested in duplicates. The diluted serum was incubated with 100-150 fluorescent focus units (FFU) of mNG SARS-CoV-2 at 37° C. for 1 h (final dilution range of 1:20 to 1:20,480), after which the serum-virus mixtures were inoculated onto the pre-seeded Vero-E6 cell monolayer in 96-well plates. After 1 h infection, the inoculum was removed and 100 μl of overlay medium (DMEM supplemented with 0.8% methylcellulose, 2% FBS, and 1% P/S) was added to each well. After incubating the plates at 37° C. for 16 h, raw images of mNG fluorescent foci were acquired using Cytation™ 7 (BioTek) armed with 2.5×FL Zeiss objective with widefield of view and processed using the software settings (GFP [469,525] threshold 4000, object selection size 50-1000 μm). The foci in each well were counted and normalized to the non-serum-treated controls to calculate the relative infectivities. The curves of the relative infectivity versus the serum dilutions (log 10 values) were plotted using Prism 9 (GraphPad). A nonlinear regression method with log (inhibitor) vs. response-variable slope (four parameters) model (bottom and top parameters were constrained to 0 and 100, respectively) was used to determine the dilution fold that neutralized 50% of mNG SARS-CoV-2 (defined as FFRNT₅₀) in GraphPad Prism 9. Each serum was tested in duplicates.

Antiviral testing. A549-hACE2 cells were used to evaluate the efficacy of a monoclonal antibody IgG14 and antiviral drug remdesivir. The sources of IgG14 and remdesivir were previously reported (Xie et al., Nat Commun 11, 5214, 2020; Ku et al., Nature Communications, 10.1038/s41467-41020-20789-41467, 2021). Briefly, A549-hACE2 cells (1.2×10⁴) were seeded in each well of black μCLEAR flat-bottom 96-well plate (Greiner Bio-one™). The cells were incubated overnight at 37° C. with 5% CO₂. For antibody testing, on the following day, IgG14 was 3-fold serially diluted and incubated with mNG Δ3678 at 37° C. for 1 h, after which the antibody-virus mixtures were inoculated into the 96-well plates that were pre-seeded A549-hACE2 cells. For antiviral testing, remdesivir was 3-fold serially diluted in DMSO and further diluted as 100 folds in the culture medium containing mNG Δ3678 virus. Fifty μl of the compound-virus mixture were immediately added to the cells at a final MOI of 1.0. At 24 h post-infection, 25 μl of Hoechst 33342 Solution (400-fold diluted in Hank's Balanced Salt Solution; Gibco) were added to each well to stain the cell nucleus. The plate was sealed with Breath-Easy sealing membrane (Diversified Biotech), incubated at 37° C. for 20 min, and quantified for mNG fluorescence on CX5 imager (ThermoFisher Scientific). The raw images (2×2 montage) were acquired using 4× objective. The total cells (indicated by nucleus staining) and mNG-positive cells were quantified for each well. Infection rates were determined by dividing the mNG-positive cell number by total cell number. Relative infection rates were obtained by normalizing the infection rates of treated groups to those of no-treated controls. The curves of the relative infection rates versus the concentration were plotted using Prism 9 (GraphPad). A nonlinear regression method was used to determine the concentration of antiviral that suppress 50% of mNG fluorescence (EC₅₀). Experiment was tested in quadruplicates.

Statistics. Hamsters and mice were randomly allocated into different groups. The investigators were not blinded to allocation during the experiments or to the outcome assessment. No statistical methods were used to predetermine sample size. Descriptive statistics have been provided in the figure legends. For in vitro replication kinetics, Kruskal-Wallis analysis of variance was conducted to detect any significant variation among replicates. If no significant variation was detected, the results were pooled for further comparison. Differences between continuous variables were assessed with a non-parametric Mann-Whitney test. The PFU and genomic copies were analyzed using an unpaired two-tailed t test. The weight loss data were shown as mean±standard deviation and statistically analyzed using two-factor analysis of variance (ANOVA) with Tukey's post hoc test. The animal survival rates were analyzed using a mixed-model ANOVA using Dunnett's test for multiple comparisons. Analyses were performed in Prism version 9.0 (GraphPad, San Diego, CA).

### Example 2

### A. Results and Discussions

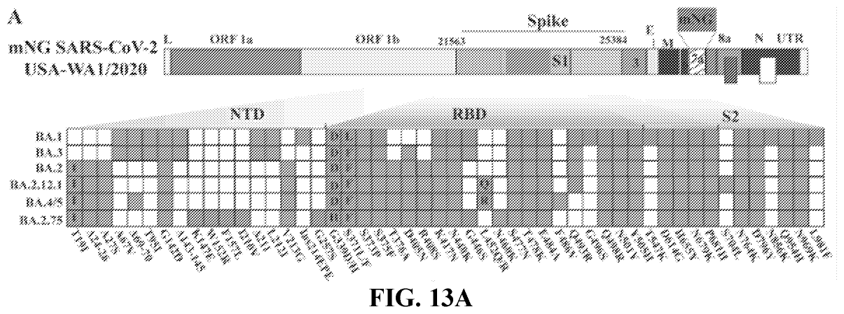

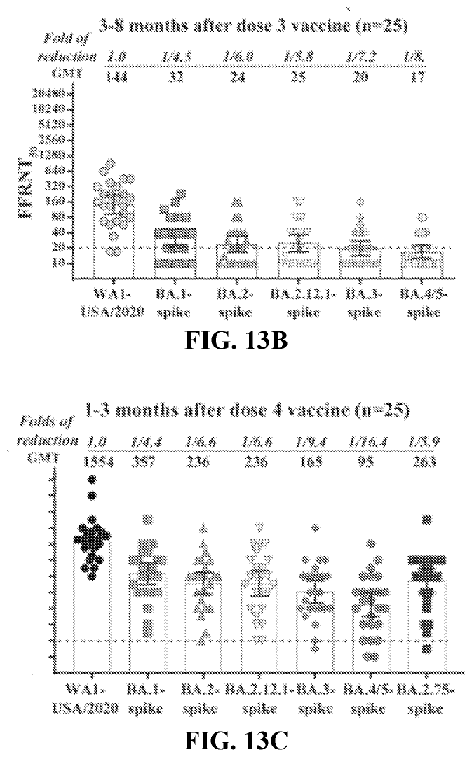

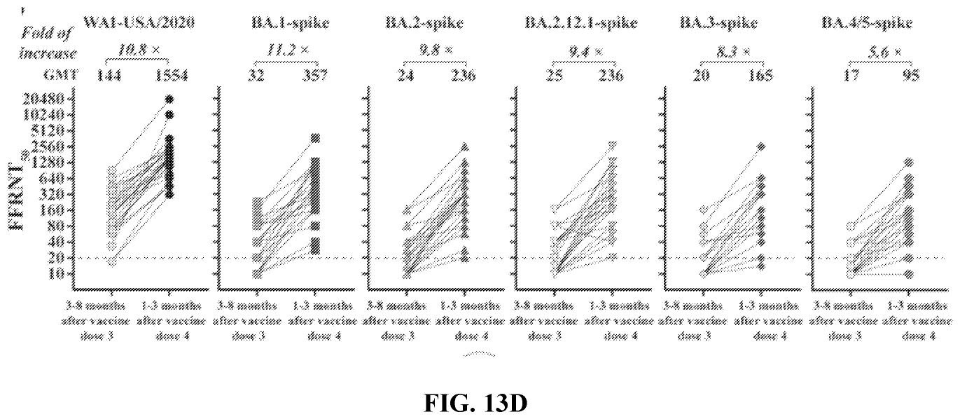

Experimental approach and rationale. A set of previously established recombinant SARS-CoV-2s were used to determine the serum neutralization against different Omicron sublineages. Each recombinant SARS-CoV-2 contained a complete spike gene from BA.1, BA.2, BA.2.12.1, BA.3, or BA.4/5 in the backbone of USA-WA1/2020 (a virus strain isolated in January 2020) containing an mNeonGreen (mNG) reporter, resulting in BA.1-, BA.2-, BA.2.12.1-, BA.3-, or BA.4/5-spike mNG SARS-CoV-2 (Kurhade et al., 2022b). BA.4 and BA.5 have an identical spike sequence and are denoted as BA.4/5. FIG. 13A summarizes the amino acid mutations of the spike protein from different Omicron sublineages. An mNeonGreen (mNG) gene was engineered into the open-reading-frame-7 (ORF7) of the viral genome to enable a fluorescent focus reduction neutralization test (FFRNT) in a high-throughput format (Zou et al., 2022a). The insertion of mNG reporter anntenuated SARS-CoV-2 replication and pathohgenesis (Johnson et al., 2022; Liu et al., 2022). The FFRNT has been reliably used to measure antibody neutralization for COVID-19 vaccine research and development (Kurhade et al., 2022a; Kurhade et al., 2022b).

Using FFRNT, the neutralization of three panels of human sera were measured against the chimeric Omicron sublineage-spike mNG SARS-CoV-2s. The first panel consisted of 25 pairs of sera collected from individuals before and after dose 4 of Pfizer or Moderna's original vaccine (Table 2). Those specimens were tested negative against viral nucleocapsid protein, suggesting those individuals had not been infected by SARS-CoV-2. The second and third serum panels were collected from individuals who had received 2 (n=29; Table 3) or 3 (n=38; Table 4) doses of the original mRNA vaccine and subsequently contracted Omicron BA.1 breakthrough infection. The BA.1 breakthrough infection was confirmed for each patient by sequencing viral RNA collected from nasopharyngeal swab samples. Tables 3-5 summarize (i) the serum information and (ii) the 50% fluorescent focus-reduction neutralization titers (FFRNT₅₀) against USA-WA1/2020, BA.1-, BA.2-, BA.2.12.1-, BA.3-, and BA.4/5-spike SARS-CoV-2s. The description and analysis of the FFRNT₅₀ results against different Omicron sublineages are detailed in the following sections for each serum panel.

The booster effect by dose 4 mRNA vaccine is less pronounced against BA.4/5 compared to USA-WA1,2020 and other omicron sublineages. To measure 4 doses of vaccine-elicited neutralization, 25 pairs of sera were collected from individuals before and after dose 4 of Pfizer or Moderna mRNA vaccine. For each serum pair, one sample was collected 3-8 months after dose 3 vaccine; the other sample was obtained from the same individual 1-3 months after dose 4 vaccine (Table 2). Before the 4^{th} dose vaccine, the 3-dose-vaccine sera neutralized USA-WA1/2020, BA.1-, BA.2-, BA.2.12.1-, BA.3-, and BA.4/5-spike mNG viruses with low geometric mean titers (GMTs) of 144, 32, 24, 25, 20, and 17, respectively (FIG. 13B); after the 4^{th} dose vaccine, the GMTs increased significantly to 1554, 357, 236, 236, 165, and 95, respectively (FIG. 13C); so, the 4^{th} dose vaccine significantly increased the neutralization against the corresponding viruses by 10.8-, 11.2-, 9.8-, 9.4-, 8.3-, and 5.6-fold, respectively (FIG. 13D). Despite the significant increase in neutralization after the 4^{th} dose vaccine, the GMTs against BA.1-, BA.2-, BA.2.12.1-, BA.3-, and BA.4/5-spike viruses were 4.4-, 6.6-, 6.6-, 9.4-, and 16.4-fold lower than the GMT against the USA-WA1/2020, respectively (FIG. 13C). These results support three conclusions. First, among the tested Omicron sublineages, BA.5 possesses the greatest evasion of vaccine-elicited neutralization. The results are in agreement with other studies supporting that BA.5 and other Omicron sublineages efficiently evade vaccine-elicited neutralization (Arora et al., 2022; Cao et al., 2022; Hachmann et al., 2022; Sheward et al., 2022; Tan et al., 2022). Second, the booster effect by the 4^{th} dose is less pronounced against BA.4/5 compared to USA-WA1/2020 and other omicron sublineages. It should be noted that dose 4 did increase the neutralizing GMT against BA.4/5 from 17 (FIG. 13B) to 95 (FIG. 13C). A recent study reported a neutralizing titer of 70 as the threshold to prevent breakthrough infections of Delta variant (Zou et al., 2022b). Although the minimal neutralizing titer required to prevent BA.5 infection has not been determined, the low neutralization against BA.5 after dose 3 vaccine [GMT of 103 at 1-month post dose 3, reported by Kurhade et al. (Kurhade et al., 2022b)] and dose 4 vaccine (GMT of 95 at 1- to 3-month post dose 4, reported here), together with the increased viral transmissibility, could account for the ongoing surge of BA.5 around the world. Third, an updated vaccine that matches the highly immune-evasive and prevalent BA.5 spike is needed to mitigate the current and future Omicron surges. The results support the U.S. Food and Drug Administration's recommendation to include BA.5 spike for future COVID-19 vaccine booster doses.

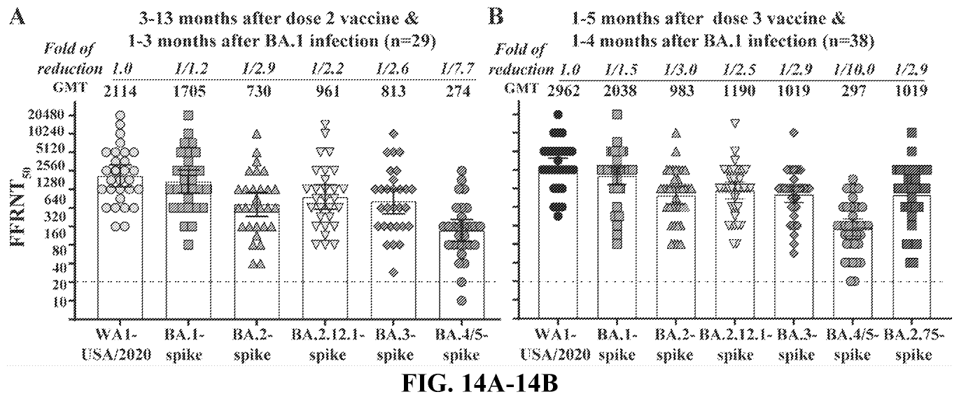

High neutralization against BA.5 and other Omicron sublineages after 2 or 3 doses of vaccine plus BA.1 infection. To compare with 4-dose-vaccine sera, we measured the neutralization against Omicron sublineages using sera collected from individuals who had received 2 or 3 doses of the original mRNA vaccine and subsequently contracted BA.1 infection (FIG. 14). Tables 4 and 5 summarize the FFRNT₅₀ results for 2-dose-vaccine-plus-BA.1-infection sera and 3-dose-vaccine-plus-BA.1-infection sera, respectively. The 2-dose-vaccine-plus-BA.1-infection sera neutralized BA.1, BA.2, BA.2.12.1, BA.3, and BA.4/5 with GMTs of 2114, 1705, 730, 961, 813, and 274, respectively (FIG. 14A); the 3-dose-vaccine-plus-BA.1-infection sera showed slightly higher GMTs of 2962, 2038, 983, 1190, 1019, and 297, respectively (FIG. 14B). So, the GMT ratios between the 3-dose-vaccine-plus-BA. 1-infection sera and 2-dose-vaccine-plus-BA.1-infection sera were 1.4, 1.2, 1.3, 1.2, 1.3, and 1.1 when neutralizing USA-WA 1/2020, BA.1-, BA.2-, BA.2.12.1-, BA.3-, and BA.4/5-spike viruses, respectively; these GMT differences between the two serum groups were statistically insignificant, suggesting the extra dose of vaccine does not significantly boost neutralization for the 3-dose-vaccine-plus-BA.1-infection sera.

In contrast, the GMT ratios between the 2-dose-vaccine-plus-BA.1-infection and 4-dose-vaccine sera were 1.4, 4.8, 3.1, 4.1, 4.9, and 3.9 when neutralizing USA-WA1/2020, BA. 1-, BA.2-, BA.2.12.1-, BA.3-, and BA.4/5-spike viruses, respectively. The result suggests that, compared with the two extra doses of vaccine in the 4-dose-vaccine sera, the BA.1 infection in the 2-dose-vaccine-plus-BA.1-infection sera is more efficient in boosting both the magnitude and breadth of neutralization against all Omicron sublineages; however, the neutralization against BA.5 was still the lowest among all tested sublineages.

For the 2-dose-vaccine-plus-BA.1-infection sera, the GMTs against BA.1-, BA.2-, BA.2.12.1-, BA.3-, and BA.4/5-spike viruses were 1.2-, 2.9-, 2.2-, 2.6-, and 7.7-fold lower than the GMT against the USA-WA1/2020, respectively (FIG. 14A); similar results were observed for the 3-dose-vaccine-plus-BA.1-infection sera, with GMTs against BA.1-, BA.2-, BA.2.12.1-, BA.3-, and BA.4/5-spike viruses that were 1.5-, 3.0-, 2.5-, 2.9-, and 10-fold lower than the GMT against the USA-WA1/2020, respectively (FIG. 14B). The GMT decreases against Omicron sublineages for the 2-dose-vaccine-plus-BA.1-infection sera and those for the 3-dose-vaccine-plus-BA.1-infection sera are significantly less than those observed for the 4-dose-vaccine sera (Compare FIGS. 14A and 14B with FIG. 13C). The results again indicate that BA.1 infection of vaccinated people efficiently boosts the breadth of neutralization against all tested Omicron sublineages. However, such BA.1 infection-mediated boost of neutralizing magnitude/breadth is dependent on previous vaccination. This is because BA.1 infection of unvaccinated people did not elicit greater neutralizing magnitude/breadth against Omicron sublineages than 3 doses of mRNA vaccine (Kurhade et al., 2022b).

Neutralization against Omicron sublineage BA.2.75. To assess the neutralization of the newly emerged Omicron sublineage BA.2.75, the complete spike gene of BA.2.75 (FIG. 13A) was engineered into the backbone of mNG USA-WA1/2020, resulting in BA.2.75-spike mNG SARS-CoV-2. The BA.2.75-spike mNG SARS-CoV-2 was sequenced to ensure no undesired mutations. When tested with 4-dose-vaccine sera, the neutralizing GMT against BA.2.75-spike virus was 2.8-fold higher than that against BA.5-spike virus (FIG. 13C). Similarly, when tested with 3-dose-vaccine-plus-BA.1-infection sera, the neutralizing GMT against BA.2.75-spike virus was 3.4-fold higher than that against BA.5-spike virus (FIG. 14B). Collectively, the results indicate that BA.2.75 is less immune-evasive than BA.5.

TABLE 2
Twenty-five pairs of human serum samples collected after
dose 3 and 4 of mRNA vaccine, Related to FIG. 13.

| | | | | | Serum | Serum | Interval | |
|---|---|---|---|---|---|---|---|---|
| | | | | | collection | collection | days | |
| | | | | | time (days | time (days | between | mRNA |
| Serum | Pair | Age | Gender | | post-dose 3 | post-dose 4 | dose 3 & 4 | Vaccine |
| ID | # | (year) | (F/M) | Ethnicity | vaccination) | vaccination) | vaccination | type |
| 1 | 1 | 62 | F | White | 174 | | 174 | Pfizer |
| 2 | | | | | | 37 | | |
| 3 | 2 | 84 | M | White | 186 | | 217 | Pfizer |
| 4 | | | | | | 26 | | |
| 5 | 3 | 80 | M | Hispanic | 184 | | 196 | Pfizer |
| 6 | | | | | | 30 | | |
| 7 | 4 | 78 | M | White | 184 | | 209 | Pfizer |
| 8 | | | | | | 56 | | |
| 9 | 5 | 87 | M | White | 243 | | 247 | Pfizer |
| 10 | | | | | | 24 | | |
| 11 | 6 | 66 | F | White | 184 | | 241 | Pfizer |
| 12 | | | | | | 48 | | |
| 13 | 7 | 83 | F | White | 148 | | 222 | Pfizer |
| 14 | | | | | | 23 | | |
| 15 | 8 | 84 | F | White | 174 | | 209 | Pfizer |
| 16 | | | | | | 55 | | |
| 17 | 9 | 86 | M | White | 189 | | 215 | Pfizer |
| 18 | | | | | | 51 | | |
| 19 | 10 | 87 | F | Hispanic | 195 | | 259 | Pfizer |
| 20 | | | | | | 43 | | |
| 21 | 11 | 67 | F | Black | 186 | | 233 | Pfizer |
| 22 | | | | | | 50 | | |
| 23 | 12 | 86 | M | White | 156 | | 217 | Pfizer |
| 24 | | | | | | 49 | | |
| 25 | 13 | 80 | M | Black | 184 | | 240 | Pfizer |
| 26 | | | | | | 44 | | |
| 27 | 14 | 72 | F | White | 191 | | 230 | Pfizer |
| 28 | | | | | | 52 | | |
| 29 | 15 | 75 | F | White | 143 | | 209 | Pfizer |
| 30 | | | | | | 78 | | |
| 31 | 16 | 73 | M | White | 163 | | 192 | Moderna |
| 32 | | | | | | 94 | | |
| 33 | 17 | 75 | M | Black | 198 | | 207 | Pfizer |
| 34 | | | | | | 47 | | |
| 35 | 18 | 80 | M | White | 146 | | 205 | Pfizer |
| 36 | | | | | | 52 | | |
| 37 | 19 | 78 | M | White | 196 | | 214 | Pfizer |
| 38 | | | | | | 73 | | |
| 39 | 20 | 92 | F | White | 163 | | 228 | Pfizer |
| 40 | | | | | | 27 | | |
| 41 | 21 | 59 | F | Hispanic | 203 | | 254 | Pfizer |
| 42 | | | | | | 27 | | (dose 1-3) |
| | | | | | | | | Moderna |
| | | | | | | | | (dose 4) |
| 43 | 22 | 90 | M | Black | 231 | | 231 | Pfizer |
| 44 | | | | | | 34 | | |
| 45 | 23 | 94 | F | White | 110 | | 228 | Pfizer |
| 46 | | | | | | 47 | | |
| 47 | 24 | 71 | F | White | 143 | | 243 | Moderna |
| 48 | | | | | | 27 | | (dose 1-3) |
| | | | | | | | | Pfizer |
| | | | | | | | | (dose 4) |
| 49 | 25 | 84 | F | White | 235 | | 257 | Pfizer |
| 50 | | | | | | 35 | | |
| ^{#}GMT PD3 | — | — | — | — | — | — | — | — |
| ^{†}95% CI | | | | | | | | |
| PD3 | | | | | | | | |
| GMT PD4 | — | — | — | — | — | — | — | — |
| 95% CI | | | | | | | | |
| PD4 | | | | | | | | |

| | *FFRNT₅₀ | | | | | | |
|---|---|---|---|---|---|---|---|
| Serum | USA- | BA.1- | BA.2- | BA.2.12. | BA.3- | BA.4/5- | BA.2.75- |
| ID | WA1/2020 | spike | spike | 1-spike | spike | spike | spike |
| 1 | 17 | \hat{ }10 | 10 | 10 | 10 | 10 | — |
| 2 | 320 | 40 | 28 | 20 | 20 | 10 | 40 |
| 3 | 17 | 10 | 10 | 10 | 10 | 10 | — |
| 4 | 640 | 160 | 80 | 80 | 80 | 20 | 80 |
| 5 | 34 | 10 | 10 | 10 | 10 | 10 | — |
| 6 | 10240 | 453 | 640 | 905 | 320 | 320 | 640 |
| 7 | 57 | 10 | 10 | 10 | 10 | 10 | — |
| 8 | 453 | 28 | 20 | 20 | 14 | 10 | 14 |
| 9 | 57 | 10 | 10 | 10 | 10 | 10 | — |
| 10 | 1810 | 1280 | 453 | 640 | 453 | 113 | 640 |
| 11 | 67 | 20 | 10 | 10 | 10 | 10 | — |
| 12 | 453 | 160 | 80 | 40 | 40 | 20 | 28 |
| 13 | 67 | 10 | 10 | 10 | 10 | 10 | — |
| 14 | 1522 | 320 | 320 | 320 | 160 | 160 | 320 |
| 15 | 80 | 10 | 20 | 14 | 10 | 10 | — |
| 16 | 1076 | 160 | 226 | 160 | 113 | 80 | 160 |
| 17 | 95 | 40 | 28 | 40 | 20 | 10 | — |
| 18 | 1522 | 640 | 320 | 320 | 320 | 20 | 640 |
| 19 | 135 | 40 | 20 | 40 | 20 | 20 | — |
| 20 | 1076 | 320 | 320 | 226 | 160 | 160 | 113 |
| 21 | 135 | 20 | 10 | 10 | 10 | 10 | — |
| 22 | 1522 | 160 | 160 | 226 | 80 | 80 | 160 |
| 23 | 135 | 40 | 40 | 80 | 20 | 40 | — |
| 24 | 1810 | 640 | 320 | 320 | 160 | 320 | 320 |
| 25 | 160 | 10 | 10 | 10 | 10 | 10 | — |
| 26 | 1280 | 640 | 320 | 453 | 320 | 160 | 320 |
| 27 | 190 | 20 | 14 | 10 | 10 | 10 | — |
| 28 | 1522 | 1280 | 320 | 453 | 320 | 40 | 453 |
| 29 | 190 | 40 | 28 | 28 | 20 | 14 | — |
| 30 | 2560 | 905 | 905 | 1280 | 640 | 453 | 640 |
| 31 | 226 | 40 | 20 | 80 | 40 | 40 | — |
| 32 | 640 | 80 | 57 | 40 | 57 | 40 | 40 |
| 33 | 226 | 80 | 40 | 40 | 40 | 40 | — |
| 34 | 2153 | 453 | 113 | 57 | 80 | 57 | 640 |
| 35 | 226 | 80 | 80 | 20 | 20 | 14 | — |
| 36 | 3620 | 1280 | 1280 | 1280 | 640 | 640 | 640 |
| 37 | 320 | 80 | 40 | 40 | 40 | 20 | — |
| 38 | 761 | 226 | 80 | 80 | 80 | 40 | 320 |
| 39 | 320 | 113 | 80 | 80 | 40 | 20 | — |
| 40 | 2153 | 160 | 160 | 160 | 80 | 113 | 640 |
| 41 | 381 | 40 | 20 | 20 | 20 | 10 | — |
| 42 | 1810 | 640 | 640 | 1280 | 320 | 160 | 453 |
| 43 | 453 | 80 | 40 | 40 | 57 | 20 | — |
| 44 | 2560 | 453 | 320 | 320 | 320 | 160 | 320 |
| 45 | 453 | 40 | 40 | 40 | 20 | 40 | — |
| 46 | 1280 | 320 | 160 | 160 | 80 | 80 | 320 |
| 47 | 640 | 226 | 160 | 160 | 160 | 80 | — |
| 48 | 2153 | 905 | 640 | 640 | 640 | 320 | 640 |
| 49 | 905 | 160 | 160 | 160 | 80 | 80 | — |
| 50 | 20480 | 3620 | 2560 | 2560 | 2560 | 1280 | 3620 |
| ^{#}GMT PD3 | 144 | 32 | 24 | 25 | 20 | 17 | — |
| ^{†}95% CI | 93-220 | 22-48 | 17-34 | 17-37 | 14-27 | 13-23 | — |
| PD3 | | | | | | | |
| GMT PD4 | 1554 | 357 | 236 | 236 | 165 | 95 | 263 |
| 95% CI | 1069-2261 | 224-569 | 147-379 | 136-409 | 101-268 | 56-160 | 157-441 |
| PD4 | | | | | | | |

*Individual FFRNT₅₀ value is the geometric mean of duplicate plaque assay results.
\hat{ }FFRNT₅₀ of <20 was treated as 10 for plot purposes and statistical analysis.
^{#}Geometric mean neutralizing titers (GMT).
^{†}95% confidence interval (95% CI) for the GMT.

TABLE 3
Twenty-nine human serum samples collected after 2 doses of mRNA vaccine
and a subsequent Omicron BA.1 breakthrough infection, Related to FIG. 14.

| | | | | | COVID test positive | Serum collection time |
|---|---|---|---|---|---|---|
| Sample | Age | Gender | | Vaccine | days post last | (Post COVID test |
| ID | (year) | (M/F) | Ethnicity | type | vaccine | positive days) |
| 1 | 37 | F | White | Moderna | 285 | 59 |
| 2 | 32 | M | Hispanic | Pfizer | 187 | 73 |
| 3 | 20 | M | Hispanic | Moderna | 401 | 48 |
| 4 | 96 | F | Hispanic | Pfizer | 341 | 50 |
| 5 | 87 | M | White | Moderna | 350 | 51 |
| 6 | 36 | F | White | Pfizer | 191 | 71 |
| 7 | 31 | F | Black | Pfizer | 124 | 58 |
| 8 | 51 | F | White | Moderna | 165 | 15 |
| 9 | 57 | M | Asian | Pfizer | 392 | 30 |
| 10 | 78 | M | Black | Moderna | 152 | 34 |
| 11 | 64 | F | Black | Pfizer | 232 | 35 |
| 12 | 30 | M | Asian | Pfizer | 214 | 64 |
| 13 | 74 | F | White | Pfizer | 389 | 34 |
| 14 | 21 | F | Hispanic | Moderna | 254 | 49 |
| 15 | 31 | F | White | Pfizer | 317 | 105 |
| 16 | 62 | M | White | Pfizer | 316 | 29 |
| 17 | 38 | F | Asian | Pfizer | 98 | 33 |
| 18 | 30 | F | Hispanic | Pfizer | 126 | 43 |
| 19 | 33 | F | Asian | Moderna | 326 | 48 |
| 20 | 28 | M | Hispanic | Pfizer | 159 | 60 |
| 21 | 74 | M | White | Pfizer | 313 | 118 |
| 22 | 66 | M | White | Pfizer | 156 | 16 |
| 23 | 59 | M | White | Pfizer | 239 | 18 |
| 24 | 18 | F | Hispanic | Pfizer | 309 | 18 |
| 25 | 35 | M | White | Moderna | 303 | 63 |
| 26 | 63 | F | Hispanic | Pfizer | 279 | 36 |
| 27 | 51 | M | Asian | Pfizer | 391 | 28 |
| 28 | 71 | F | White | Pfizer | 346 | 39 |
| 29 | 46 | F | Hispanic | Pfizer | 266 | 29 |
| ^{#}GMT | — | — | — | — | — | — |
| ^{†}95% CI | — | — | — | — | — | — |

| | *FFRNT₅₀ | | | | | |
|---|---|---|---|---|---|---|
| Sample | USA- | BA.1- | BA.2- | BA.2.12.1- | BA.3- | BA.4/5- |
| ID | WA1/2020 | spike | spike | spike | spike | spike |
| 1 | 320 | 160 | 80 | 160 | 57 | 20 |
| 2 | 320 | 320 | 80 | 160 | 160 | 80 |
| 3 | 640 | 640 | 320 | 320 | 320 | 160 |
| 4 | 640 | 640 | 226 | 320 | 320 | 113 |
| 5 | 640 | 640 | 160 | 226 | 160 | 40 |
| 6 | 640 | 320 | 160 | 160 | 160 | 80 |
| 7 | 905 | 640 | 320 | 320 | 320 | 320 |
| 8 | 1280 | 1280 | 640 | 905 | 640 | 226 |
| 9 | 1280 | 1280 | 320 | 905 | 640 | 160 |
| 10 | 1280 | 1810 | 905 | 1280 | 1280 | 320 |
| 11 | 1280 | 1280 | 640 | 905 | 640 | 320 |
| 12 | 1280 | 905 | 320 | 453 | 320 | 160 |
| 13 | 1810 | 1280 | 320 | 453 | 320 | 160 |
| 14 | 1810 | 2560 | 1280 | 1280 | 1280 | 226 |
| 15 | 1810 | 1280 | 640 | 640 | 640 | 320 |
| 16 | 2560 | 1810 | 640 | 640 | 1280 | 320 |
| 17 | 2560 | 2560 | 1280 | 1280 | 1280 | 320 |
| 18 | 2560 | 2560 | 1280 | 1280 | 1280 | 453 |
| 19 | 2560 | 2560 | 1280 | 1810 | 1280 | 320 |
| 20 | 3620 | 640 | 640 | 640 | 320 | 160 |
| 21 | 3620 | 1280 | 640 | 640 | 640 | 160 |
| 22 | 5120 | 3620 | 1280 | 2560 | 1280 | 640 |
| 23 | 5120 | 7241 | 2560 | 5120 | 5120 | 2560 |
| 24 | 5120 | 5120 | 1280 | 2560 | 1810 | 640 |
| 25 | 5120 | 5120 | 2560 | 1280 | 2560 | 320 |
| 26 | 7241 | 5120 | 2560 | 2560 | 2560 | 1280 |
| 27 | 10240 | 10240 | 5120 | 10240 | 5120 | 1810 |
| 28 | 14482 | 7241 | 3620 | 5120 | 5120 | 640 |
| 29 | 20480 | 20480 | 10240 | 14482 | 10240 | 1280 |
| ^{#}GMT | 2114 | 1705 | 730 | 961 | 813 | 274 |
| ^{†}95% CI | 1411-3167 | 1112-2614 | 465-1145 | 612-1509 | 511-1294 | 182-412 |

*Individual FFRNT₅₀ value is the geometric mean of duplicate plaque assay results.
^{#}Geometric mean neutralizing titers (GMT).
^{†}95% confidence interval (95% CI) for the GMT.

TABLE 4
Thirty-eight human serum samples collected after 3 doses of mRNA vaccine and
a subsequent Omicron BA. 1 breakthrough infection, Related to FIG. 14.

| | | | | | COVID test positive | Serum collection time |
|---|---|---|---|---|---|---|
| Sample | Age | Gender | | Vaccine | days post last | (Post COVID test |
| ID | (year) | (M/F) | Ethnicity | type | vaccine | positive days) |
| 1 | 36 | M | Hispanic | Pfizer | 142 | 91 |
| 2 | 36 | M | Hispanic | Pfizer | 142 | 61 |
| 3 | 64 | M | White | Pfizer | 40 | 75 |
| 4 | 62 | F | White | Pfizer | 130 | 81 |
| 5 | 27 | M | White | Moderna | 111 | 134 |
| 6 | 34 | F | Black | Pfizer | 85 | 40 |
| 7 | 60 | F | Hispanic | Pfizer | 39 | 76 |
| 8 | 29 | F | White | Pfizer | 15 | 91 |
| 9 | 58 | M | Asian | Pfizer | 30 | 113 |
| 10 | 39 | F | Asian | Pfizer | 110 | 23 |
| 11 | 72 | M | White | Pfizer | 28 | 32 |
| 12 | 43 | F | Asian | Pfizer | 89 | 58 |
| 13 | 45 | F | Hispanic | Pfizer | 141 | 46 |
| 14 | 51 | F | Black | Pfizer | 44 | 35 |
| 15 | 56 | F | Black | Pfizer | 98 | 93 |
| 16 | 67 | F | White | Pfizer | 71 | 63 |
| 17 | 64 | M | White | Pfizer | 114 | 33 |
| 18 | 28 | F | White | Pfizer | 158 | 61 |
| 19 | 64 | M | Asian | Pfizer | 117 | 73 |
| 20 | 63 | F | White | Pfizer | 174 | 22 |
| 21 | 41 | F | Hispanic | Moderna | 84 | 64 |
| | | | | (dose 1-2) & | | |
| | | | | Pfizer | | |
| | | | | (dose 3) | | |
| 22 | 64 | F | Asian | Pfizer | 118 | 105 |
| 23 | 54 | M | White | Pfizer | 93 | 34 |
| 24 | 26 | F | White | Moderna | 63 | 51 |
| 25 | 44 | F | Hispanic | Pfizer | 43 | 15 |
| 26 | 77 | M | Hispanic | Pfizer | 130 | 64 |
| 27 | 69 | M | White | Pfizer | 124 | 79 |
| 28 | 73 | F | White | Moderna | 164 | 36 |
| 29 | 84 | F | Hispanic | Pfizer | 99 | 46 |
| 30 | 61 | F | White | Pfizer | 169 | 40 |
| 31 | 58 | M | Hispanic | Moderna | 77 | 73 |
| 32 | 75 | M | White | Pfizer | 131 | 108 |
| 33 | 79 | F | White | Pfizer | 144 | 17 |
| 34 | 38 | M | White | Pfizer | 109 | 49 |
| 35 | 71 | M | White | Pfizer | 129 | 54 |
| 36 | 39 | F | Hispanic | Pfizer | 118 | 126 |
| 37 | 68 | M | White | Pfizer | 125 | 71 |
| 38 | 66 | M | White | Pfizer | 96 | 41 |
| ^{#}GMT | — | — | — | — | — | — |
| ^{†}95% CI | — | — | — | — | — | — |

| | FFRNT₅₀ | | | | | | |
|---|---|---|---|---|---|---|---|
| Sample | USA- | BA.1- | BA.2- | BA.2.12.1- | BA.3- | BA.4/5- | BA.2.75- |
| ID | WA1/2020 | spike | spike | spike | spike | spike | spike |
| 1 | 453 | 226 | 160 | 160 | 160 | 40 | 160 |
| 2 | 640 | 453 | 160 | 320 | 320 | 40 | 160 |
| 3 | 640 | 160 | 160 | 160 | 113 | 80 | 80 |
| 4 | 640 | 453 | 320 | 320 | 320 | 160 | 160 |
| 5 | 640 | 320 | 160 | 320 | 226 | 80 | 160 |
| 6 | 1280 | 1280 | 640 | 640 | 640 | 320 | 640 |
| 7 | 1280 | 640 | 320 | 320 | 320 | 80 | 453 |
| 8 | 1280 | 453 | 320 | 320 | 320 | 80 | 320 |
| 9 | 1280 | 1280 | 640 | 1280 | 640 | 453 | 640 |
| 10 | 2560 | 2560 | 1280 | 1280 | 1280 | 320 | 1280 |
| 11 | 2560 | 2560 | 1280 | 1810 | 1280 | 226 | 2560 |
| 12 | 2560 | 2560 | 2560 | 1810 | 1280 | 905 | 2560 |
| 13 | 2560 | 5120 | 1810 | 2560 | 2560 | 640 | 1810 |
| 14 | 2560 | 2560 | 905 | 640 | 1280 | 320 | 1280 |
| 15 | 2560 | 2560 | 640 | 1280 | 1280 | 226 | 1280 |
| 16 | 2560 | 1280 | 640 | 640 | 640 | 160 | 640 |
| 17 | 2560 | 2560 | 1280 | 1280 | 1280 | 320 | 905 |
| 18 | 2560 | 2560 | 1280 | 1280 | 1280 | 320 | 1810 |
| 19 | 2560 | 2560 | 1280 | 1280 | 1280 | 160 | 2560 |
| 20 | 2560 | 2560 | 1280 | 2560 | 905 | 1280 | 1280 |
| 21 | 2560 | 1280 | 320 | 453 | 453 | 160 | 640 |
| 22 | 3620 | 2560 | 1280 | 1280 | 1280 | 320 | 2560 |
| 23 | 5120 | 2560 | 2560 | 2560 | 1810 | 640 | 2560 |
| 24 | 5120 | 5120 | 1810 | 2560 | 2560 | 1280 | 2560 |
| 25 | 5120 | 2560 | 1280 | 1280 | 1280 | 640 | 1810 |
| 26 | 5120 | 5120 | 2560 | 2560 | 2560 | 1280 | 2560 |
| 27 | 5120 | 2560 | 1280 | 1810 | 1280 | 320 | 1280 |
| 28 | 5120 | 3620 | 2560 | 1810 | 1280 | 226 | 640 |
| 29 | 5120 | 5120 | 1280 | 2560 | 1280 | 226 | 2560 |
| 30 | 5120 | 2560 | 640 | 1280 | 905 | 226 | 1280 |
| 31 | 5120 | 1810 | 640 | 905 | 1280 | 320 | 1280 |
| 32 | 5120 | 2560 | 1280 | 1280 | 1280 | 320 | 1280 |
| 33 | 5120 | 2560 | 640 | 1280 | 905 | 640 | 1280 |
| 34 | 10240 | 5120 | 5120 | 5120 | 2560 | 1280 | 2560 |
| 35 | 10240 | 7241 | 2560 | 2560 | 2560 | 80 | 5120 |
| 36 | 10240 | 5120 | 2560 | 3620 | 2560 | 640 | 2560 |
| 37 | 10240 | 5120 | 2560 | 2560 | 2560 | 640 | 80 |
| 38 | 20480 | 20480 | 10240 | 14482 | 10240 | 1810 | 10240 |
| ^{#}GMT | 2962 | 2038 | 983 | 1190 | 1019 | 297 | 1019 |
| ^{†}95% CI | 2212-3967 | 1462-2841 | 712-1357 | 869-1630 | 759-1367 | 216-410 | 702-1480 |

*Individual FFRNT₅₀ value is the geometric mean of duplicate plaque assay results.
^{#}Geometric mean neutralizing titers (GMT).
^{†}95% confidence interval (95% CI) for the GMT.

### B. Methods

Ethical statement. The work was performed in a biosafety level 3 (BSL-3) laboratory with redundant fans in the biosafety cabinets at The University of Texas Medical Branch at Galveston. All personnel wore powered air-purifying respirators (Breathe Easy, 3M) with Tyvek suits, aprons, booties, and double gloves.

The research protocol regarding the use of human serum specimens was reviewed and approved by the University of Texas Medical Branch (UTMB) Institutional Review Board (IRB number 20-0070). No informed consent was required since these deidentified sera were leftover specimens before being discarded. No diagnosis or treatment was involved.

Cells. Vero E6 (ATCC® CRL-1586) was purchased from the American Type Culture Collection (ATCC, Bethesda, MD), and maintained in a high-glucose Dulbecco's modified Eagle's medium (DMEM) containing 10% fetal bovine serum (FBS; HyClone Laboratories, South Logan, UT) and 1% penicillin/streptomycin at 37° C. with 5% CO₂. Culture media and antibiotics were purchased from ThermoFisher Scientific (Waltham, MA). The cell line was tested negative for mycoplasma.

Human Serum. Three panels of human sera were used in the study. The first panel consisted of 25 pairs of sera collected from individuals 3-8 months after vaccine dose 3, and no more than 3 months after dose 4 of Pfizer or Moderna vaccine. This panel had been tested negative for SARS-CoV-2 nucleocapsid protein expression using Bio-Plex Pro Human IgG SARS-CoV-2 N/RBD/S1/S2 4-Plex Panel (Bio-rad). The second serum panel (n=29) was collected from individuals who had received 2 doses of mRNA vaccine and subsequently contracted Omicron BA.1. The third serum panel (n=38) was collected from individuals who had received 3 doses of mRNA vaccine and subsequently contracted Omicron BA.1. The genotype of infecting virus was verified by the molecular tests with FDA's Emergency Use Authorization and Sanger sequencing. The de-identified human sera were heat-inactivated at 56° C. for 30 min before the neutralization test. The serum information is presented in Table S1-3.

Recombinant Omicron sublineage spike mNG SARS-CoV-2. Recombinant Omicron sublineage BA.1-, BA.2-, BA.2.12.1-, BA.3-, BA.4/5-spike mNG SARS-CoV-2s that was constructed by engineering the complete spike gene from the indicated variants into an infectious cDNA clone of mNG USA-WA1/2020 were reported previously (Kurhade et al., 2022b; Xie et al., 2020). BA.2.75-spike sequence was based on GISAID EPI_ISL_13521499. FIG. 13A depicts the spike mutations from different Omicron sublineages. The full-length cDNA of viral genome bearing the variant spike was assembled via in vitro ligation and used as a template for in vitro transcription. The full-length viral RNA was then electroporated into Vero E6-TMPRSS2 cells. On day 3-4 post electroporation, the original P0 virus was harvested from the electroporated cells and propagated for another round on Vero E6 cells to produce the P1 virus. The infectious titer of the P1 virus was quantified by fluorescent focus assay on Vero E6 cells and sequenced for the complete spike gene to ensure no undesired mutations. The P1 virus was used for the neutralization test. The protocols for the mutagenesis of mNG SARS-CoV-2 and virus production were reported previously (Hachmann et al., N Engl J Med 387, 86-88, 2022).

Fluorescent focus reduction neutralization test. A fluorescent focus reduction neutralization test (FFRNT) was performed to measure the neutralization titers of sera against USA-WA1/2020, BA.1-, BA.2-, BA.2.12.1-, BA.3-, and BA4/5-spike mNG SARS-CoV-2. The FFRNT protocol was reported previously (Zou et al., 2022a). Vero E6 cells were seeded onto 96-well plates with 2.5×10⁴ cells per well (Greiner Bio-one™) and incubated overnight. On the next day, each serum was 2-fold serially diluted in a culture medium and mixed with 100-150 focus-forming units of mNG SARS-CoV-2. The final serum dilution ranged from 1:20 to 1:20,480. After incubation at 37° C. for 1 h, the serum-virus mixtures were loaded onto the pre-seeded Vero E6 cell monolayer in 96-well plates. After 1 h infection, the inoculum was removed and 100 μl of overlay medium containing 0.8% methylcellulose was added to each well. After incubating the plates at 37° C. for 16 h, raw images of mNG foci were acquired using Cytation*r 7 (BioTek) armed with 2.5×FL Zeiss objective with a wide field of view and processed using the software settings (GFP [469,525] threshold 4000, object selection size 50-1000 μm). The fluorescent mNG foci were counted in each well and normalized to the non-serum-treated controls to calculate the relative infectivities. The FFRNT₅₀ value was defined as the minimal serum dilution to suppress >50% of fluorescent foci. The neutralization titer of each serum was determined in duplicate assays, and the geometric mean was taken. Tables 3-5 summarize the FFRNT₅₀ results.

1. An attenuated recombinant SARS-CoV-2 comprising a SARS-CoV-2 genome having (i) a transcriptional regulatory sequences (TRS) comprising a nucleotide sequence of CCGGAT and (ii) a deletion of open reading frames 3, 6, 7, and 8.

2. The attenuated recombinant SARS-CoV-2 of claim 1, wherein the nucleic acid segment encoding the attenuated recombinant SARS-CoV-2 has a nucleic acid sequence that is at least 98% identical to the nucleic acid sequence of SEQ ID NO:1.

3. The attenuated recombinant SARS-CoV-2 of claim 1, wherein the nucleic acid encoding the attenuated recombinant SARS-CoV-2 has a nucleic acid sequence of SEQ ID NO:1.

4. The attenuated recombinant SARS-CoV-2 of claim 1, further comprising a heterologous S protein.

5. The attenuated recombinant SARS-CoV-2 of claim 4, wherein the heterologous S protein is a S protein variant.

6. The attenuated recombinant SARS-CoV-2 of claim 1, is a SARS-CoV-2 variant

7. The attenuated recombinant SARS-CoV-2 of claim 1, wherein the nucleic acid encoding the attenuated recombinant SARS-CoV-2 is comprised in an expression cassette.

8. The attenuated recombinant SARS-CoV-2 of claim 7, wherein the expression cassette is comprised in a plasmid backbone.

9. The attenuated recombinant SARS-CoV-2 of claim 1, further comprising a nucleic acid segment encoding a reporter protein.

10. The attenuated recombinant SARS-CoV-2 of claim 1, wherein the reporter protein is a fluorescent or luminescent protein.

11. The attenuated recombinant SARS-CoV-2 of claim 10, wherein the fluorescent protein is mNeonGreen protein.

12. The attenuated recombinant SARS-CoV-2 of claim 10, wherein the luminescent protein is nanoluciferase protein.

13. A host cell comprising the attenuated recombinant SARS-CoV-2 of any one of claims 1 to 12.

14. A vaccine composition comprising the attenuated recombinant SARS-CoV-2 of any one of claims 1 to 12.

15. An assay for SARS-CoV-2 replication comprising:
contacting a cultured cell expressing or containing a SARS-CoV-2 nucleotide sequence of any one of claims 1 to 10 forming a test cell;
contacting the test cell with a test agent; and
assessing the replication of the SARS-CoV-2 in the presence of the test agent.

16. The assay of claim 15, wherein the cultured cell is a Vero cell.

17. The assay of claim 15, wherein the cultured cell is assayed in a multi-well plate.

18. The assay of claim 17, wherein the multi-well plate is a 96 well microtiter plate.

19. The assay of claim 15, wherein the cultured cells are incubated for about 12, 24, 36, or 48 hours before measuring the reporter signal.
